(19) United States
(12) Patent Application Publication
(10) Pub. No.: US 2022/0000733 A1
(43) Pub. Date: Jan. 6, 2022

(54) ALCOHOL-BASED SANITIZER FORMULATION FOR TOPICAL APPLICATION

(71) Applicant: MARY KAY INC., Addison, TX (US)

(72) Inventor: Wanli ZHAO, Addison, TX (US)

(21) Appl. No.: 17/363,815

(22) Filed: Jun. 30, 2021

Related U.S. Application Data
(60) Provisional application No. 63/046,397, filed on Jun. 30, 2020.
(60) Provisional application No. 63/110,748, filed on Nov. 6, 2020.

(51) Int. Cl. A61K 8/34; A61K 8/44; A61K 8/92; A61K 8/9794; A61Q 19/00; A61Q 19/10; A61Q 17/00

(52) U.S. Cl. CPC A61K 8/345; A61K 8/44; A61K 8/922; A61Q 17/005; A61Q 19/007; A61Q 19/10; A61K 8/34; A61K 8/9794

(57) ABSTRACT

Disclosed is are methods and compositions capable of sanitizing and hydrating skin. The compositions can include alcohol, glycerin, betaine, PEG-50 shea butter, butylene glycol, and Aloe barbadensis leaf extract.

This application claims priority to U.S. Provisional Application No. 63/046,397 filed Jun. 30, 2020, and U.S. Provisional Application No. 63/110,748 filed Nov. 6, 2020. The contents of the referenced applications are incorporated into the present application by reference.

### BACKGROUND OF THE INVENTION

### A. Field of the Invention

The present invention relates generally to topical compositions and methods that can be used to sanitize skin and/or hydrate skin. In one aspect, the compositions can include alcohol, glycerin, betaine, PEG-50 shea butter, butylene glycol, and Aloe barbadensis leaf extract.

### B. Description of Related Art

Various factors can lead to skin dryness, including weather, heat, harsh chemicals such as skin sanitizers, and other extrinsic and intrinsic factors which may damage skin and cause skin dryness, to name a few examples. These stresses can change the visual appearance, physical properties, or physiological functions of skin and tissue in ways that are considered visually undesirable. Notable and obvious changes include coarse surface texture, itching, flaking, scaling, peeling, fine lines or cracks, and/or redness.

Maintaining moisture of the skin helps overcome some unwanted changes in skin. However, maintaining moisture of the skin can be difficult. This is especially true for subjects with skin that is more dry than average (dry skin type). As an example, alcohol-based skin sanitizers that contain at least 60% alcohol are useful for ridding the skin of potential pathogens, thereby sanitizing the skin, but these sanitizers can strip the skin of its natural, protective oils. Frequent use of some alcohol-based skin sanitizers can lead to and exacerbate dry skin issues and/or cause skin irritation or erythema.

Others have attempted to create compositions and methods that sanitize skin. However, many attempts have been ineffective, only addressed one or a few of the undesired outcomes, or caused unacceptable side effects themselves.

### SUMMARY OF THE INVENTION

A solution to at least one or more of the aforementioned problems associated with current hand-sanitizer products has been discovered. In one aspect, a solution of the present invention can be directed to a combination of ingredients that can effectively sanitize skin and/or hydrate and/or moisturize skin. The combination of ingredients can include alcohol, glycerin, betaine, PEG-50 shea butter, butylene glycol, and Aloe barbadensis leaf extract. The combination of ingredients can be used in particular amounts. An additional benefit of the compositions and methods of the present invention when compared with existing hand sanitizer products is that the compositions and methods of the present invention can effectively sanitize skin without leading to or worsening dry skin. The compositions and methods of the present invention can also reduce or mitigate unwanted side effects such as skin irritation or erythema, which are typically associated with the use of hand sanitizers.

In some aspects, there is disclosed a topical composition that includes any one of, any combination of, or all of alcohol, glycerin, betaine, PEG-50 shea butter, butylene glycol, and Aloe barbadensis leaf extract. In some aspects, there is disclosed a topical composition that includes, consists essentially of, or consists of any one of, any combination of, or all of alcohol, glycerin, betaine, PEG-50 shea butter, butylene glycol, and/or Aloe barbadensis leaf extract. The amounts of the ingredients within the composition can vary (e.g., amounts can be as low as 0.000001% to as high as 99% w/w or any range therein). In some aspects, the topical composition includes 60 to 80% by weight of alcohol, 0.1% to 15% by weight of glycerin, 0.1% to 10% by weight of betaine, 0.001% to 1% by weight of PEG-50 shea butter, 0.001 to 1% by weight of butylene glycol, and 0.001 to 1% by weight of Aloe barbadensis leaf extract. In some instances, the composition includes 60 to 70% by weight of alcohol. In some instances, the composition includes 20 to 30% by weight of water. In some instances, the composition includes 1 to 10% by weight of glycerin. In some instances, the alcohol includes ethanol and/or denatured alcohol. In some instances, the denatured alcohol includes tert-butyl alcohol and/or denatonium benzoate. In some instances, the denatured alcohol includes specifically denatured alcohol 40-B.

In some instances, the composition includes an effective amount of alcohol, glycerin, betaine, PEG-50 shea butter, butylene glycol, and Aloe barbadensis leaf extract, wherein topical application of the composition sanitizes skin and/or hydrates skin. In some instances, the composition includes an effective amount of alcohol to sanitize skin. In some instances, composition includes an effective amount of glycerin, betaine, PEG-50 shea butter, butylene glycol, and Aloe barbadensis leaf extract to hydrate skin.

In some instances, the composition further includes one or more of a humectant, a skin conditioning agent, a viscosity controlling agent, a preservative, and/or a pH adjuster. In some particular aspects, the composition of the present invention can include a preservative (e.g., phenoxyethanol). Data confirms that compositions of the present invention have anti-microbial properties with and without the inclusion of a preservative. Therefore, and in some aspects, the compositions of the present invention may not include a preservative or an additional preservative. In some instances, the composition further includes water and/or phenoxyethanol. In some instances, the composition includes 10 to 40% by weight water and 0.0001 to 0.1% by weight phenoxyethanol.

In some aspects, the compositions disclosed herein are used to hydrate skin and/or sanitize skin. In some aspects, the compositions disclosed herein are used to better hydrate and/or sanitize skin compared to other skin sanitizing products. In some aspects, the compositions disclosed herein are used to boost moisture levels on top layers of skin. In some aspects, the compositions disclosed herein are used to sanitize the top layers of skin. In some aspects, the compositions disclosed herein can be used to sanitize by killing bacteria, viruses, fungi, etc. In some aspects, the compositions disclosed herein are anti-bacterial, anti-viral, and/or anti-fungal sanitizers. In some aspects, the compositions disclosed herein are sanitizers having at least one of, any combination of, or all of bactericidal, virucidal, and fungicidal properties. In some instances, topical application of the composition to skin of a person reduces one or more microorganism populations present on the skin by at least 99.9% within 15 seconds of application of the composition. Non-limiting examples of microorganism populations that can be reduced by at least 99.9% within 15 seconds include Haemophilus influenza, Streptococcus pneumoniae (such as ATCC Number 6303), Bacteroides fragilis, Pseudomonas aeruginosa (such as ATCC Number 27853), Campylobacter jejuni, Streptococcus pyogenes, Streptococcus pneumoniae (such as ATCC Number 49619), Enterobacter cloacae, Burkholderia cepacia, Escherichia coli, Klebsiella pneumoniae, Pseudomonas aeruginosa (such as ATCC Number 15442), Serratia marcescens, Salmonella enterica, Shigella sonnei, Enterococcus faecalis, Staphylococcus aureus, methicillin-resistant Staphylococcus aureus, and/or Listeria monocytogenes. In some instances, topical application of the composition to the skin of a person reduces one or more microorganism populations present on the skin by at least 99.99% within 15 seconds of application of the composition. Non-limiting examples of microorganism populations that can be reduced by at least 99.99% within 15 seconds include Bacteroides fragilis, Pseudomonas aeruginosa (such as ATCC Number 27853), Campylobacter jejuni, Streptococcus pyogenes, or Streptococcus pneumoniae (such as ATCC Number 49619), Enterobacter cloacae, Burkholderia cepacia, Escherichia coli, Klebsiella pneumoniae, Pseudomonas aeruginosa (such as ATCC Number 15442), Serratia marcescens, Salmonella enterica, Shigella sonnei, Enterococcus faecalis, Staphylococcus aureus, methicillin-resistant Staphylococcus aureus, and/or Listeria monocytogenes. In some instances, topical application of the composition to the skin of a person reduces one or more microorganism populations present on the skin by at least 99.999% within 15 seconds of application of the composition. Non-limiting examples of microorganism populations that can be reduced by at least 99.999% within 15 seconds include comprise Enterobacter cloacae, Burkholderia cepacia, Escherichia coli, Klebsiella pneumoniae, Pseudomonas aeruginosa (such as ATCC Number 15442), Serratia marcescens, Salmonella enterica, Shigella sonnei, Enterococcus faecalis, Staphylococcus aureus, methicillin-resistant Staphylococcus aureus, and/or Listeria monocytogenes.

In some instances, the method comprises topically applying any one of the compositions disclosed herein to skin in need thereof. In one aspect, any one of the compositions disclosed herein are topically applied and the composition is left on the application area, removed from the application area after a period of time, and/or removed directly after application.

In some aspects, the composition can be applied to a person's hands and can dry within 120 seconds, within 90 seconds, within 60 seconds, within 45 seconds, within 30 seconds, within 15 seconds, within 10 seconds, or within 5 seconds after application to the person's hands. In some aspects, the composition can be applied to a person's hands and can dry 1 second to 45 seconds after application to hands, or 5 seconds to 45 seconds after application to hands, or 10 seconds to 30 seconds after application to hands. In some instances, the alcohol present in the composition can be volatile, which can allow for the relatively quick drying times after topical application. In addition to alcohol, other volatile components can be added to further aid in the composition drying after topical application to hands.

In some aspects, the composition is applied to skin multiple times per day. In some instances, the composition can be applied to skin 3 to 20 times per day. In some instances, the composition can be applied to skin 3 times per day. In some instances, the composition can be applied to skin 4 times per day. In some instances, the composition can be applied to skin 5 times per day. In some instances, the composition can be applied to skin 6 times per day. In some instances, the composition can be applied to skin 7 times per day. In some instances, the composition can be applied to skin more than 7 times per day. In some instances, the composition is applied to skin of the hands. In some instances, the composition is applied to the skin of the hands, arms, face, feet, legs, waist, abdomen, neck, face, and/or head. In some instances, the composition is applied to the finger and/or toe nails.

In some aspects, the composition is applied to clean skin. In some instances, the composition is left on the skin to be absorbed. In some instances, one or more skin care compositions are applied to the skin before application of the composition to the skin. In some instances, one or more skin care compositions are applied to the skin after application of the composition to the skin. In some instances, the application of the composition is followed by application of a moisturizer. In some instances, the moisturizer is applied after the composition is absorbed into the skin. In some aspects, the composition can be combined with one or more additional compositions for treating skin.

It is also contemplated that the compositions disclosed throughout this specification can be used as a leave-on or rinse-off composition. By way of example, a leave-on composition can be one that is topically applied to skin and remains on the skin for a period of time (e.g., at least 1, 2, 3, 4, 5, 6, 7, 8, 9, 10, 20, or 30 minutes, or at least 1, 2, 3, 4, 5, 6, 7, 8, 9, 10, 11, 12, 13, 14, 15, 16, 17, 18, 19, 20, 21, 22, 23 or 24 hours, or overnight or throughout the day). Alternatively, a rinse-off composition can be a product that is intended to be applied to the skin and then removed or rinsed from the skin (e.g., with water) within a period of time such as less than 5, 4, 3, 2, or 1 minute. In some instances, the composition is designed to be washed away after 30 seconds, 1 minutes, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 20 minutes, 30 minutes, 40 minutes, 50 minutes, 60 minutes, or any amount or range therein. An example of a rinse off composition can be a skin cleanser, shampoo, conditioner, or soap. An example of a leave-on composition can be a skin sanitizer, moisturizer, sunscreen, mask, overnight cream, or a day cream.

In some aspects, the compositions of the present invention can further include a surfactant, a silicone containing compounds, a UV agent, an oil, and/or other ingredients identified in this specification or those known in the art. The composition can be a liquid, lotion, cream, body butter, mask, scrub, wash, gel, serum, emulsion (e.g., oil-in-water, water-in-oil, silicone-in-water, water-in-silicone, water-in-oil-in-water, oil-in-water-in-oil, oil-in-water-in-silicone, etc.), solutions (e.g., aqueous or hydro-alcoholic solutions), anhydrous base (e.g., lipstick or a powder), ointments, milk, paste, aerosol, solid forms, eye jellies, gel serums, gel emulsions, etc. In some instances, the composition is a serum, a cream, a gel, a cream gel, an oil-in-water emulsion, a water-in-oil emulsion, or a liquid. In some instances, the composition is a liquid. In some instances, the composition is comprised in an ampule. The composition can be formulated for topical skin application at least 1, 2, 3, 4, 5, 6, 7, or more times a day during use. In some aspects of the present invention, compositions can be storage stable or color stable, or both. It is also contemplated that the viscosity of the composition can be selected to achieve a desired result, e.g., depending on the type of composition desired, the viscosity of such composition can be from about 1 cps to well over 1 million cps or any range or integer derivable therein (e.g., 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps, etc., as measured on a Brookfield Viscometer using a TC spindle at 2.5 rpm at 25° C.).

The compositions, in non-limiting aspects, can have a pH of about 6 to about 9. In some aspects, the pH can be 1, 2, 3, 4, 5, 6, 7, 8, 9, 10, 11, 12, 13, or 14. The compositions can include a triglyceride. Non-limiting examples include small, medium, and large chain triglycerides. In certain aspects, the triglyceride is a medium chain triglyceride (e.g., caprylic capric triglyceride). The compositions can also include preservatives. Non-limiting examples of preservatives include phenoxyethanol, methylparaben, propylparaben, iodopropynyl butylcarbamate, potassium sorbate, sodium benzoate, or any mixture thereof. In some embodiments, the composition is paraben-free.

Compositions of the present invention can have UVA and UVB absorption properties. The compositions can have a sun protection factor (SPF) of 2, 3, 4, 5, 6, 7, 8, 9, 10, 11, 12, 13, 14, 15, 20, 25, 30, 35, 40, 45, 50, 55, 60, or more, or any integer or derivative therein. The compositions can be sunscreen lotions, sprays, or creams.

The compositions of the present invention can also include any one of, any combination of, or all of the following additional ingredients: a conditioning agent, a moisturizing agent, a pH adjuster, a structuring agent, inorganic salts, a preservative, a thickening agent, a silicone containing compound, an essential oil, a fragrance, a vitamin, a pharmaceutical ingredient, or an antioxidant, or any combination of such ingredients or mixtures of such ingredients. In certain aspects, the composition can include at least two, three, four, five, six, seven, eight, nine, ten, or more, or all of these additional ingredients identified in the previous sentence. Non-limiting examples of these additional ingredients are identified throughout this specification and are incorporated into this section by reference. The amounts of such ingredients can range from 0.0001% to 99.9% by weight or volume of the composition, or any integer or range in between as disclosed in other sections of this specification, which are incorporated into this paragraph by reference.

Kits that include the compositions of the present invention are also contemplated. In certain embodiments, the composition is comprised in a container. The container can be a bottle, dispenser, or package. The container can dispense a pre-determined amount of the composition. In certain aspects, the compositions is dispensed in a spray, mist, dollop, or liquid. The container can include indicia on its surface. The indicia can be a word, an abbreviation, a picture, or a symbol.

It is contemplated that any embodiment discussed in this specification can be implemented with respect to any method or composition of the invention, and vice versa. Furthermore, compositions of the invention can be used to achieve methods of the invention.

In some embodiments, compositions of the present invention can be pharmaceutically or cosmetically elegant or can have pleasant tactile properties. “Pharmaceutically elegant,” “cosmetically elegant,” and/or “pleasant tactile properties” describes a composition that has particular tactile properties which feel pleasant on the skin (e.g., compositions that are not too watery or greasy, compositions that have a silky texture, compositions that are non-tacky or sticky, etc.). Pharmaceutically or cosmetically elegant can also relate to the creaminess or lubricity properties of the composition or to the moisture retaining properties of the composition.

Also contemplated is a product comprising a composition of the present invention. In non-limiting aspects, the product can be a cosmetic product. The cosmetic product can be those described in other sections of this specification or those known to a person of skill in the art. Non-limiting examples of products include a moisturizer, a cream, a lotion, a skin softener, a serum, a gel, a wash, a body butter, a scrub, a foundation, a night cream, a lipstick, a cleanser, a toner, a sunscreen, a mask, an anti-aging product, a deodorant, an antiperspirant, a perfume, a cologne, etc.

Also disclosed are the following Embodiments 1 to 38 of the present invention. Embodiment 1 is a method of sanitizing and hydrating skin, the method comprising topically applying to skin of a person a composition comprising an effective amount of alcohol, glycerin, betaine, PEG-50 shea butter, butylene glycol, and Aloe barbadensis leaf extract, wherein topical application of the composition sanitizes and hydrates skin. Embodiment 2 is the method of Embodiment 1, wherein topical application of the composition to the skin of a person reduces one or more microorganism populations present on the skin by at least 99.9% within 15 seconds of application of the composition. Embodiment 3 is the method of Embodiments 1 to 2, wherein topical application of the composition to the skin of a person reduces one or more microorganism populations present on the skin by at least 99.99% within 15 seconds of application of the composition. Embodiment 4 is the method of any of Embodiments 1 to 3, wherein topical application of the composition to the skin of a person reduces one or more microorganism populations present on the skin by at least 99.999% within 15 seconds of application of the composition. Embodiment 5 is the method of any of Embodiments 2 to 4, wherein the one or more microorganism populations capable of being reduced comprise Haemophilus influenza, Streptococcus pneumoniae, Bacteroides fragilis, Pseudomonas aeruginosa, Campylobacter jejuni, Streptococcus pyogenes, Enterobacter cloacae, Burkholderia cepacia, Escherichia coli, Klebsiella pneumoniae, Serratia marcescens, Salmonella enterica, Shigella sonnei, Enterococcus faecalis, Staphylococcus aureus, methicillin-resistant Staphylococcus aureus, and/or Listeria monocytogenes. Embodiment 6 is the method of any of Embodiments 1 to 5, wherein the composition comprises 60 to 80% by weight of alcohol, 0.1% to 15% by weight of glycerin, 0.1% to 10% by weight of betaine, 0.001% to 1% by weight of PEG-50 shea butter, 0.001 to 1% by weight of butylene glycol, and 0.001 to 1% by weight of Aloe barbadensis leaf extract. Embodiment 7 is the method of any of Embodiments 1 to 6, wherein the composition comprises an effective amount of alcohol to sanitize skin. Embodiment 8 is the method of any of Embodiments 1 to 7, wherein the composition comprises an effective amount of glycerin, betaine, PEG-50 shea butter, butylene glycol, and Aloe barbadensis leaf extract to hydrate skin. Embodiment 9 is the method of any of Embodiments 1 to 8, wherein the composition further comprises one or more of: a humectant, a skin conditioning agent, a viscosity controlling agent, a preservative, and/or a pH adjuster. Embodiment 10 is the method of any of Embodiments 1 to 9, wherein the composition further comprises water and/or phenoxyethanol. Embodiment 11 is the method of any of Embodiments 1 to 10, wherein the composition comprises 10 to 40% by weight water and 0.0001 to 0.1% by weight phenoxyethanol. Embodiment 12 is the method of any of Embodiments 1 to 11, wherein the composition comprises 60 to 70% by weight of alcohol. Embodiment 13 is the method of any of Embodiments 1 to 12, wherein the composition comprises 20 to 30% by weight of water. Embodiment 14 is the method of any of Embodiments 1 to 13, wherein the composition comprises 1 to 10% by weight of glycerin. Embodiment 15 is the method of any of Embodiments 1 to 14, wherein the alcohol comprises ethanol and/or denatured alcohol. Embodiment 16 is the method of any of Embodiments 1 to 15, wherein the alcohol comprises tert-Butyl alcohol or t-butyl alcohol and denatonium benzoate. Embodiment 17 is the method of any one of Embodiments 1 to 16, wherein the composition comprises 60 to 80% by weight of alcohol, 20 wt. % to 30 wt. % water, 3% to 7% by weight of glycerin, 0.5% to 1.5% by weight of betaine, 0.01% to 0.1% by weight of PEG-50 shea butter, 0.01 to 0.1% by weight of butylene glycol, 0.005 to 0.05% by weight of Aloe barbadensis leaf extract, and 0.0005 wt. % to 0.002 wt. % of phenoxyethanol. Embodiment 18 is the method of any of Embodiments 1 to 17, wherein the composition consists of alcohol, water, glycerin, betaine, PEG-50 shea butter, butylene glycol, Aloe barbadensis leaf extract, and phenoxyethanol.

Embodiment 19 is a topical sanitizing and hydrating composition comprising an effective amount of a combination of alcohol, glycerin, betaine, PEG-50 shea butter, butylene glycol, and Aloe barbadensis leaf extract to sanitize skin and/or hydrate skin. Embodiment 20 is the composition of Embodiment 19, wherein the composition is capable of reducing one or more microorganism populations present on the skin by at least 99.9% within 15 seconds of application of the composition to the skin. Embodiment 21 is the composition of Embodiments 19 to 20, wherein the composition is capable of reducing one or more microorganism populations present on the skin by at least 99.99% within 15 seconds of application of the composition. Embodiment 22 is the composition of Embodiments 19 to 21, wherein the composition is capable of reducing one or more microorganism populations present on the skin by at least 99.999% within 15 seconds of application of the composition. Embodiment 23 is the composition of Embodiments 20 to 22, wherein the one or more microorganism populations capable of being reduced comprise Haemophilus influenza, Streptococcus pneumoniae, Bacteroides fragilis, Pseudomonas aeruginosa, Campylobacter jejuni, Streptococcus pyogenes, Enterobacter cloacae, Burkholderia cepacia, Escherichia coli, Klebsiella pneumoniae, Serratia marcescens, Salmonella enterica, Shigella sonnei, Enterococcus faecalis, Staphylococcus aureus, methicillin-resistant Staphylococcus aureus, and/or Listeria monocytogenes. Embodiment 24 is the composition of Embodiments 19 to 23, wherein the composition comprises 60 to 80% by weight of alcohol, 0.1% to 15% by weight of glycerin, 0.1% to 10% by weight of betaine, 0.001% to 1% by weight of PEG-50 shea butter, 0.001 to 1% by weight of butylene glycol, and 0.001 to 1% by weight of Aloe barbadensis leaf extract. Embodiment 25 is the composition of Embodiments 19 to 24, wherein the composition comprises an effective amount of alcohol to sanitize skin. Embodiment 26 is the composition of Embodiments 19 to 25, wherein the composition comprises an effective amount of glycerin, betaine, PEG-50 shea butter, butylene glycol, and Aloe barbadensis leaf extract to hydrate skin. Embodiment 27 is the composition of Embodiments 19 to 26, wherein the composition further comprises one or more of: a humectant, a skin conditioning agent, a viscosity controlling agent, a preservative, and/or a pH adjuster. Embodiment 28 is the composition of Embodiments 19 to 27, wherein the composition further comprises water and/or phenoxyethanol. Embodiment 29 is the composition of Embodiments 19 to 28, wherein the composition comprises 10 to 40% by weight water and 0.0001 to 0.1% by weight phenoxyethanol. Embodiment 30 is the composition of Embodiments 19 to 29, wherein the composition comprises 60 to 70% by weight of alcohol. Embodiment 31 is the composition of Embodiments 19 to 30, wherein the composition comprises 20 to 30% by weight of water. Embodiment 32 is the composition of Embodiments 19 to 31, wherein the composition comprises 1 to 10% by weight of glycerin. Embodiment 33 is the composition of Embodiments 19 to 32, wherein the alcohol comprises tert-butyl alcohol and denatonium benzoate. Embodiment 34 is the composition of Embodiments 19 to 33, wherein the alcohol comprises ethanol and/or denatured alcohol. Embodiment 35 is the composition of Embodiments 19 to 34, wherein the composition comprises 60 to 80% by weight of alcohol, 20 wt. % to 30 wt. % water, 3% to 7% by weight of glycerin, 0.5% to 1.5% by weight of betaine, 0.01% to 0.1% by weight of PEG-50 shea butter, 0.01 to 0.1% by weight of butylene glycol, 0.005 to 0.05% by weight of Aloe barbadensis leaf extract, and 0.0005 wt. % to 0.002 wt. % of phenoxyethanol. Embodiment 36 is the composition of embodiments 19 to 35, wherein the composition consists of alcohol, water, glycerin, betaine, PEG-50 shea butter, butylene glycol, Aloe barbadensis leaf extract, and phenoxyethanol. Embodiment 37 is the composition of Embodiments 19 to 36, wherein the composition is capable of drying within 120 seconds, within 90 seconds, within 60 seconds, within 45 seconds, within 30 seconds, within 15 seconds, within 10 seconds, or within 5 seconds, or 1 to 120 seconds, or 1 to 60 seconds, or 10 to 60 seconds, or 10 to 45 seconds, or 10 to 30 seconds, after topical application to skin.

Embodiment 38 is a method of sanitizing skin and/or hydrating skin, the method comprising topically applying to skin of a person a composition of any one of claims 19 to 37, wherein topical application of the composition sanitizes skin and/or hydrates skin.

“Topical application” means to apply or spread a composition onto the surface of lips or keratinous tissue. “Topical skin composition” includes compositions suitable for topical application on skin and/or keratinous tissue. Such compositions are typically dermatologically-acceptable in that they do not have undue toxicity, incompatibility, instability, allergic response, and the like, when applied to skin and/or keratinous tissue. Topical skin care compositions of the present invention can have a selected viscosity to avoid significant dripping or pooling after application to skin and/or keratinous tissue.

“Keratinous tissue” includes keratin-containing layers disposed as the outermost protective covering of mammals and includes, but is not limited to, lips, skin, hair, and nails.

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The term “substantially” and its variations refers to ranges within 10%, within 5%, within 1%, or within 0.5%.

The terms “inhibiting” or “reducing” or any variation of these terms includes any measurable decrease or complete inhibition to achieve a desired result. The terms “promote” or “increase” or any variation of these terms includes any measurable increase, such as a measurable increase of a protein or molecule (e.g., matrix proteins such as fibronectin, laminin, collagen, or elastin or molecules such as hyaluronic acid) to achieve a desired result.

The term “effective,” as that term is used in the specification and/or claims, means adequate to accomplish a desired, expected, or intended result.

The use of the word “a” or “an” when used in conjunction with the terms “comprising,” “including,” “having,” or “containing,” or any variations of these terms, in the claims and/or the specification may mean “one,” but it is also consistent with the meaning of “one or more,” “at least one,” and “one or more than one.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. With respect to the phrase “consisting essentially of,” a basic and novel property of the compositions and methods of the present invention is a composition that can sanitize and/or hydrate skin. The composition can include alcohol, glycerin, betaine, PEG-50 shea butter, butylene glycol, and Aloe barbadensis leaf extract.

Other objects, features, and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

In one aspect of the present invention, a solution is provided to at least one or more of the problems associated with current topical products used to sanitize hands. The solution can include the use of a combination of alcohol, glycerin, betaine, PEG-50 shea butter, butylene glycol, and Aloe barbadensis leaf extract to sanitize skin and/or improve skin hydration. In some embodiments, an effective amount of a composition that includes any one of, any combination of, or all of alcohol, glycerin, betaine, PEG-50 shea butter, butylene glycol, and/or Aloe barbadensis leaf extract was found to sanitize and/or hydrate skin. In some aspects, the compositions of the present invention can include a preservative (e.g., phenoxyethanol). Data confirms that the compositions of the present invention have anti-microbial properties with and without the presence of a preservative. Therefore, and in some aspects, the compositions of the present invention may not include a preservative or may not include an additional preservative.

A particular composition of the present invention is designed to work as a topical composition. The composition relies on a unique combination of any one of, any combination of, or all of alcohol, glycerin, betaine, PEG-50 shea butter, butylene glycol, and/or Aloe barbadensis leaf extract. These combinations can be used to create topical compositions that sanitize skin and/or improve skin hydration. Non-limiting examples of such compositions are provided in Table 1 of Example 1 below.

These and other non-limiting aspects of the present invention are described in the following sections.

### A. Active Ingredients

Alcohol can be used in skin formulations to sanitize, provide a quick-drying finish, and/or immediately degrease skin. With respect to sanitizing activity, alcohols, such as ethanol, denatured alcohol, isopropyl alcohol, etc. have a nonspecific mode of action, including mainly denaturation and coagulation of proteins. Pathogenic cells can be lysed and cellular metabolism can be disrupted.

Alcohol can have a strong immediate bactericidal activity that can be observed at 30% and higher concentrations. According to the tentative final monograph for health care antiseptic products, alcohol is considered to be generally effective at between 60 and 95%. Kampf & Kramer, “Epidemiologic Background of Hand Hygiene and Evaluation of the Most Important Agents for Scrubs and Rubs,” Clin Microbiol Rev. 2004 October; 17(4): 863-893. The spectrum of bactericidal activity of ethanol is broad. In addition, alcohol has broad activity against most fungi, including yeasts and dermatophytes, at different exposure times and under different test conditions. Most naked viruses are inactivated by alcohol as well. No acquired resistance to alcohol has been reported to date. Further, alcohols are considered to be among the safest antiseptics available and generally have no toxic effect on human skin. Different formulations based on various alcohols have been tested on intact skin for 6 days and 4 weeks and have been found to be well tolerated. The skin barrier remains intact, dermal hydration does not change significantly, and the dermal sebum content remains unchanged. Kampf & Kramer, “Epidemiologic Background of Hand Hygiene and Evaluation of the Most Important Agents for Scrubs and Rubs,” Clin Microbiol Rev. 2004 October; 17(4): 863-893. Repeated exposure to alcohol or a moderately formulated product can cause or maintain skin dryness and irritation. However, and as discussed above, one of the solutions offered by the compositions of the present invention is that it can include high amounts of alcohol (e.g., 60 wt. % to 80 wt. %) and act to sanitize the skin while also reducing or avoiding skin dryness and irritation.

Alcohols used in topical applications can be denatured. Denatured alcohol is ethanol that has additives to make it poisonous, bad-tasting, foul-smelling, or nauseating to discourage recreational consumption. “Final Report of the Safety Assessment of Alcohol Denat., Including SD Alcohol 3-A, SD Alcohol 30, SD Alcohol 39, SD Alcohol 39-B, SD Alcohol 39-C, SD Alcohol 40, SD Alcohol 40-B, and SD Alcohol 40-C, and the Denaturants, Quassin, Brucine Sulfate/Brucine, and Denatonium Benzoate,” Int J Toxicol. 2008; 27 Suppl 1:1-43. Denaturing alcohol does not chemically alter the ethanol molecule unlike the denaturation process of bio-macromolecules. A specially denatured alcohol (SDA) is one of many types of denatured alcohol and is a combination of ethanol and another chemical substance, for example ethyl acetate, sodium salicylate, salicylic acid, diethyl phthalate, pyridine, methanol, tert-butyl alcohol, quassin, brucine, brucine sulfate, or essential oils. As an example, SDA 40-B is denatured with tert-butyl alcohol or t-butyl alcohol and denatonium benzoate. tert-Butyl alcohol is a clear liquid or a colorless solid, depending on the ambient temperature, with a camphor-like odor. It is soluble in water and miscible with ethanol and diethyl ether. Denatonium benzoate is a white, odorless granule soluble in water and alcohol with an unpleasantly bitter taste.

Glycerin, sometimes called glycerol or glycerine, is a naturally-occurring alcohol compound found in animal and vegetable lipids. It can be derived from natural substances by hydrolysis of fats and by fermentation of sugars; it also can be synthetically manufactured. Glycerin is a colorless, odorless, viscous liquid which freezes to a gummy paste and has a high boiling point. It is soluble in water or alcohol. Glycerin is a skin-replenishing and skin-restoring ingredient found naturally in skin and so can help to establish normal balance and hydration. Fluhr et al., “Glycerol and the skin: holistic approach to its origin and functions,” British J. Derm. 2008; 159(1): 23-34. It is one of the many substances in skin that helps maintain a healthy look and feel, defending against dryness and working to maintain skin's moisture level. Glycerin can be used in cosmetics as a humectant or emollient to improve skin's smoothness and moisture content.

Betaine, also known as trimethylglycine or glycine betaine, is a synthetic or naturally-derived ingredient that provides benefits such as increased hydration, skin protection, and enhanced texture of formulations. It is a small trimethylated aminoacid existing in a zwitterionic form at neutral pH. Synthetically produced betaine, such as cocamidopropyl betaine, functions as a surfactant. Natural betaine functions as an osmolyte, an ingredient that helps skin adapt to moisture losses and gains, essentially working to balance skin's hydration. DuPont Industrial Biosciences, “Natural Betaine in Personal Care,” 2012. Betaine also functions as a texture enhancer in cosmetics and personal care products. It is known to provide a silky feel to skin care preparations without causing tackiness. Rigano et al., Cosmetics & Toiletries 2000; 115(12): 47-54. Betaine also has the ability to increase the swelling speed of some hydrophilic thickeners. Additionally, when betaine is added to formulations containing surfactants, it has been shown to increase foam volume and reduce irritation potential of surfactants.

PEG-50 shea butter is an ethoxylated, water soluble form of whole shea butter which includes Vitamins A and E to soothe and hydrate skin while boosting collagen and Vitamin F to protect and heal skin. Malachi Oluwaseyi Israel, “Effects of Topical and Dietary Use of Shea Butter on Animals,” Am. J. Life Sci. 2014; 2(5): 303-307. It is a polyethylene glycol derivative of Butyrospermum parkii (shea butter) (q.v.) with an average of 50 moles of ethylene oxide. Shea butter is a slightly yellowish or ivory colored natural vegetable fat extracted from the nut of the African karite tree (Vitellaria paradoxa). It is used as an emollient in cosmetics and has smoothing, softening, and skin conditioning properties for dry skin. Lin et al., “Anti-Inflammatory and Skin Barrier Repair Effects of Topical Application of Some Plant Oils,” Int J Mol Sci. 2018 January; 19(1): 70.

Butylene glycol is an organic alcohol derived from petroleum and is used in cosmetics as a humectant, solvent, emollient, or conditioning agent. “Final report on the safety assessment of butylene glycol, hexylene glycol, ethoxydiglycol, and dipropylene glycol,” J. Am. College Tox. 1985; 4(5):223-248. Benefits of butylene glycol include moisture retention, dissolution of ingredients, and improved application of topical products. As a solvent, butylene glycol works well with ingredients that are not water-soluble and are difficult to dissolve. By breaking down hard-to-dissolve active ingredients, butylene glycol improves penetration, which, as a result, helps products perform more effectively. In addition to being a humectant, which binds water and pulls in hydration to the outer layer of the skin, butylene glycol can also function as an emollient by creating a barrier on the skin, which prevents water loss and softens and conditions.

Aloe barbadensis leaf extract is from the leaf of Aloe barbadensis, also known as Aloe vera. Aloe barbadensis is a stemless or very short-stemmed succulent plant growing to 60-100 cm (24-39 in) tall, spreading by offsets. The leaves are thick and fleshy, green to grey-green, with some varieties showing white flecks on their upper and lower stem surfaces. The extract can be obtained from the whole plant, the root, the flower, the stem, the leaf, the flower/leaf/stem, or the leaf/root. In particular embodiments, the leaf extract is used. In some embodiments, the extract is derived from aloe gel, the inner part of the aloe leaf. In some embodiments, the extract is an aqueous extract. In some embodiments, the leaf extract is in a 50/50 combination of butylene glycol and water. Non-limiting examples of the benefits provided by Aloe barbadensis leaf extract include moisturization, inhibition of TNF-α, providing antioxidant benefits, and/or providing anti-inflammatory benefits. “Final Report on the Safety Assessment of Aloe andongensis Extract, Aloe andongensis Leaf Juice, Aloe arborescens Leaf Extract, Aloe arborescens Leaf Juice, Aloe arborescens Leaf Protoplasts, Aloe barbadensis Flower Extract, Aloe barbadensis Leaf, Aloe barbadensis Leaf Extract, Aloe barbadensis Leaf Juice, Aloe barbadensis Leaf Polysaccharides, Aloe barbadensis Leaf Water, Aloe ferox Leaf Extract, Aloe ferox Leaf Juice, and Aloe ferox Leaf Juice Extract,” Int. J. Tox. 2007; 26 (Suppl. 2): 1-50.

This combination of ingredients can be used in different product forms to treat various skin conditions. By way of non-limiting examples, the combination of ingredients can be formulated in a liquid, an ampule, an emulsion (e.g., oil in water, water in oil), a gel, a serum, a gel emulsion, a gel serum, a lotion, a mask, a scrub, a wash, a cream, or a body butter.

The components described herein can be extracts made through extraction methods known in the art and combinations thereof. Non-limiting examples of extraction methods include the use of liquid-liquid extraction, solid phase extraction, aqueous extraction, ethyl acetate, alcohol, acetone, oil, supercritical carbon dioxide, heat, pressure, pressure drop extraction, ultrasonic extraction, etc. Extracts can be a liquid, solid, dried liquid, re-suspended solid, etc.

### B. Amounts of Ingredients

It is contemplated that the compositions of the present invention can include any amount of the ingredients discussed in this specification. The compositions can also include any number of combinations of additional ingredients described throughout this specification (e.g., pigments, or additional cosmetic or pharmaceutical ingredients). The concentrations of the any ingredient within the compositions can vary. In non-limiting embodiments, for example, the compositions can comprise, consist essentially of, or consist of, in their final form, for example, at least about 0.0001%, 0.0002%, 0.0003%, 0.0004%, 0.0005%, 0.0006%, 0.0007%, 0.0008%, 0.0009%, 0.0010%, 0.0011%, 0.0012%, 0.0013%, 0.0014%, 0.0015%, 0.0016%, 0.0017%, 0.0018%, 0.0019%, 0.0020%, 0.0021%, 0.0022%, 0.0023%, 0.0024%, 0.0025%, 0.0026%, 0.0027%, 0.0028%, 0.0029%, 0.0030%, 0.0031%, 0.0032%, 0.0033%, 0.0034%, 0.0035%, 0.0036%, 0.0037%, 0.0038%, 0.0039%, 0.0040%, 0.0041%, 0.0042%, 0.0043%, 0.0044%, 0.0045%, 0.0046%, 0.0047%, 0.0048%, 0.0049%, 0.0050%, 0.0051%, 0.0052%, 0.0053%, 0.0054%, 0.0055%, 0.0056%, 0.0057%, 0.0058%, 0.0059%, 0.0060%, 0.0061%, 0.0062%, 0.0063%, 0.0064%, 0.0065%, 0.0066%, 0.0067%, 0.0068%, 0.0069%, 0.0070%, 0.0071%, 0.0072%, 0.0073%, 0.0074%, 0.0075%, 0.0076%, 0.0077%, 0.0078%, 0.0079%, 0.0080%, 0.0081%, 0.0082%, 0.0083%, 0.0084%, 0.0085%, 0.0086%, 0.0087%, 0.0088%, 0.0089%, 0.0090%, 0.0091%, 0.0092%, 0.0093%, 0.0094%, 0.0095%, 0.0096%, 0.0097%, 0.0098%, 0.0099%, 0.0100%, 0.0200%, 0.0250%, 0.0275%, 0.0300%, 0.0325%, 0.0350%, 0.0375%, 0.0400%, 0.0425%, 0.0450%, 0.0475%, 0.0500%, 0.0525%, 0.0550%, 0.0575%, 0.0600%, 0.0625%, 0.0650%, 0.0675%, 0.0700%, 0.0725%, 0.0750%, 0.0775%, 0.0800%, 0.0825%, 0.0850%, 0.0875%, 0.0900%, 0.0925%, 0.0950%, 0.0975%, 0.1000%, 0.1250%, 0.1500%, 0.1750%, 0.2000%, 0.2250%, 0.2500%, 0.2750%, 0.3000%, 0.3250%, 0.3500%, 0.3750%, 0.4000%, 0.4250%, 0.4500%, 0.4750%, 0.5000%, 0.5250%, 0.0550%, 0.5750%, 0.6000%, 0.6250%, 0.6500%, 0.6750%, 0.7000%, 0.7250%, 0.7500%, 0.7750%, 0.8000%, 0.8250%, 0.8500%, 0.8750%, 0.9000%, 0.9250%, 0.9500%, 0.9750%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, 5.1%, 5.2%, 5.3%, 5.4%, 5.5%, 5.6%, 5.7%, 5.8%, 5.9%, 6.0%, 6.1%, 6.2%, 6.3%, 6.4%, 6.5%, 6.6%, 6.7%, 6.8%, 6.9%, 7.0%, 7.1%, 7.2%, 7.3%, 7.4%, 7.5%, 7.6%, 7.7%, 7.8%, 7.9%, 8.0%, 8.1%, 8.2%, 8.3%, 8.4%, 8.5%, 8.6%, 8.7%, 8.8%, 8.9%, 9.0%, 9.1%, 9.2%, 9.3%, 9.4%, 9.5%, 9.6%, 9.7%, 9.8%, 9.9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 60%, 65%, 70%, 75%, 80%, 85%, 90%, 95%, or 99% or any range derivable therein, of at least one of the ingredients that are mentioned throughout the specification and claims. In non-limiting aspects, the percentage can be calculated by weight or volume of the total composition. A person of ordinary skill in the art would understand that the concentrations can vary depending on the addition, substitution, and/or subtraction of ingredients in a given composition.

### C. Vehicles

The compositions of the present invention can include or be incorporated into all types of vehicles and carriers. The vehicle or carrier can be a pharmaceutically or dermatologically acceptable vehicle or carrier. Non-limiting examples of vehicles or carriers include water, glycerin, alcohol, oil, a silicon containing compound, a silicone compound, and wax. Variations and other appropriate vehicles will be apparent to the skilled artisan and are appropriate for use in the present invention. In certain aspects, the concentrations and combinations of the compounds, ingredients, and agents can be selected in such a way that the combinations are chemically compatible and do not form complexes which precipitate from the finished product.

### D. Structure

The compositions of the present invention can be structured or formulated into a variety of different forms. Non-limiting examples include liquids, emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, masks, scrubs, body butters, peels, and ointments. Variations and other structures will be apparent to the skilled artisan and are appropriate for use in the present invention.

### E. Additional Ingredients

In addition to the combination of ingredients disclosed by the inventors, the compositions can also include additional ingredients such as cosmetic ingredients and pharmaceutical active ingredients. Non-limiting examples of these additional ingredients are described in the following subsections.

1. Cosmetic Ingredients

The CTFA International Cosmetic Ingredient Dictionary and Handbook (2004 and 2008) describes a wide variety of non-limiting cosmetic ingredients that can be used in the context of the present invention. Examples of these ingredient classes include: fragrance agents (artificial and natural; e.g., gluconic acid, phenoxyethanol, and triethanolamine), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), flavoring agents/aroma agents (e.g., Stevia rebaudiana (sweetleaf) extract, and menthol), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g., A, B, C, D, E, and K), trace metals (e.g., zinc, calcium and selenium), anti-irritants (e.g., steroids and non-steroidal anti-inflammatories), botanical extracts (e.g., Aloe vera, chamomile, cucumber extract, Ginkgo biloba, ginseng, and rosemary), anti-microbial agents, antioxidants (e.g., BHT and tocopherol), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, saccharide isomerate, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, gluconolactone, calcium gluconate, cyclohexasiloxane, and dipotassium glycyrrhizate). Non-limiting examples of some of these ingredients are provided in the following subsections.

a. UV Absorption and/or Reflecting Agents

UV absorption and/or reflecting agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate (octinoxate), isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloyl trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutylphenol, and bis-ethylhexyloxyphenol methoxyphenyltriazine, 4-methylbenzylidene camphor, and isopentyl 4-methoxycinnamate. Non-limiting examples of physical sunblocks include, kaolin, talc, petrolatum and metal oxides (e.g., titanium dioxide and zinc oxide).

b. Moisturizing Agents

Non-limiting examples of moisturizing agents that can be used with the compositions of the present invention include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrrolidone carboxylic acid, potassium PCA, propylene glycol, saccharide isomerate, sodium glucuronate, sodium PCA, sorbitol, sucrose, trehalose, urea, and xylitol.

Other examples include acetylated lanolin, acetylated lanolin alcohol, alanine, algae extract, Aloe barbadensis, Aloe barbadensis extract, Aloe barbadensis gel, Althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamom (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, coco-caprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angustifolia) oil, lecithin, lemon (Citrus medica limonum) oil, linoleic acid, linolenic acid, Macadamia ternifolia nut oil, maltitol, matricaria (Chamomilla recutita) oil, methyl glucose sesquistearate, methylsilanol PCA, mineral oil, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, panthenyl ethyl ether, paraffin, PCA, peach (Prunus persica) kernel oil, peanut (Arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (Mentha piperita) oil, petrolatum, phospholipids, plankton extract, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (Santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, waxes, wheat (Triticum vulgare) germ oil, and ylang (Cananga odorata) oil.

c. Antioxidants

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

d. Structuring Agents

In other non-limiting aspects, the compositions of the present invention can include a structuring agent. Structuring agents, in certain aspects, assist in providing rheological characteristics to the composition to contribute to the composition's stability. In other aspects, structuring agents can also function as an emulsifier or surfactant. Non-limiting examples of structuring agents include sodium cocoyl glutamate, hydroxypropyl cyclodextrin, stearic acid, palmitic acid, stearyl alcohol, cetyl alcohol, behenyl alcohol, stearic acid, palmitic acid, the polyethylene glycol ether of stearyl alcohol having an average of about 1 to about 21 ethylene oxide units, the polyethylene glycol ether of cetyl alcohol having an average of about 1 to about 5 ethylene oxide units, and mixtures thereof.

e. Emulsifiers

In certain aspects of the present invention, the compositions do not include an emulsifier. In other aspects, however, the compositions can include one or more emulsifiers. Emulsifiers can reduce the interfacial tension between phases and improve the formulation and stability of an emulsion. The emulsifiers can be nonionic, cationic, anionic, and zwitterionic emulsifiers (see U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, cetearyl glucoside, cetearyl alcohol, C12-13 pareth-3, PPG-2 methyl glucose ether distearate, PPG-5-ceteth-20, bis-PEG/PPG-20/20 dimethicone, ceteth-10, polysorbate 80, cetyl phosphate, potassium cetyl phosphate, diethanolamine cetyl phosphate, polysorbate 60, glyceryl stearate, PEG-100 stearate, arachidyl alcohol, arachidyl glucoside, and mixtures thereof.

f. Silicone Containing Compounds

In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethicone, cyclomethicone, cyclohexasiloxane, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e., normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e., dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

g. Exfoliating Agent

Exfoliating agents include ingredients that remove dead skin cells on the skin's outer surface. These agents may act through mechanical, chemical, and/or other means. Non-limiting examples of mechanical exfoliating agents include abrasives such as pumice, silica, cloth, paper, shells, beads, solid crystals, solid polymers, etc. Non-limiting examples of chemical exfoliating agents include acids and enzyme exfoliants. Acids that can be used as exfoliating agents include, but are not limited to, glycolic acid, lactic acid, citric acid, a hydroxy acids, beta hydroxy acids, etc. Other exfoliating agents known to those of skill in the art are also contemplated as being useful within the context of the present invention.

h. Essential Oils

Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang. Other essential oils known to those of skill in the art are also contemplated as being useful within the context of the present invention.

i. Thickening Agents

Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene, trihydroxystearin, ammonium acryloyldimethyltaurate/VP copolymer, or a mixture of them.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., CARBOPOL™ 900 series from B. F. Goodrich).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Other useful polysaccharides include scleroglucans comprising a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

j. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), parabens (e.g., methylparabens and propylparabens), phenoxyethanol, benzyl alcohol, chlorobutanol, phenol, sorbic acid, thimerosal or combinations thereof. In some particular instances, the preservative can be phenoxyethanol.

2. Pharmaceutical Ingredients

Pharmaceutical active agents are also contemplated as being useful with the compositions of the present invention. Non-limiting examples of pharmaceutical active agents include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, anti seborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

### F. Kits

Kits are also contemplated as being used in certain aspects of the present invention. For instance, compositions of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation use, and maintain the compositions.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1

### Exemplary Formulation

A formulation having the ingredients disclosed herein was prepared as a topical skin composition. In some instances, the topical skin compositions of the present invention can be prepared as liquid, ampule, serum, cream, gel, cream gel, emulsion, or gel emulsion. The formulation in Table 1 is an example of a topical skin composition prepared as a liquid.

TABLE 1\hat{ }*

| Ingredient | % Concentration (by weight) |
|---|---|
| Denatured Alcohol | 68.03247 |
| Water | 25.88203 |
| Glycerin | 4.975 |
| Betaine | 1 |
| PEG-50 Shea Butter | 0.05 |
| Butylene Glycol | 0.0395 |
| Aloe barbadensis leaf extract | 0.02 |
| Phenoxyethanol | 0.001 |

\hat{ }Formulation can be prepared by mixing the ingredients in a beaker at room temperature or under heat, such as at 70-75° C., until homogenous. Subsequently, the formulation can be cooled to standing room temperature (20-25° C.). Further, and if desired, additional ingredients can be added, for example, to modify the rheological properties of the composition or ingredients that provide benefits to skin.
*Excipients can be added, for example, to modify the rheological properties of the composition. Alternatively, the amount of water can be varied so long as the amount of water in the composition is at least 5% w/w, and preferably between 10 to 40% w/w.

### Example 2

### In Vitro Time Kill Tests

The antimicrobial effectiveness of the Table 1 formulation was tested using time kill tests. Time kill tests provide an in vitro method for demonstrating that the antimicrobial active (denatured alcohol) of the formulation retains antimicrobial activity after incorporation into the formulation. Testing of the formulation was performed against the 31 microbial organisms listed in the Table 2 below. Tests were done in triplicate with each replicate plated in duplicate for 15 and 30 second time points, with all appropriate controls.

TABLE 2
Test Organisms

| | | ATCC | | |
|---|---|---|---|---|
| | Test Organism | Number | Contact Time | Recovery Media |
| 1 | Haemophilus influenzae | 33391 | 15 and 30 sec | Chocolate Agar |
| 2 | Bacterokies fragilis | 25285 | 15 and 30 sec | Blood Agar 5% Sheep blood |
| 3 | Enterobacter species | 13047 | 15 and 30 sec | Brain Heart Infusion Agar |
| 4 | Burkholderia cepacia | 25416 | 15 and 30 sec | Tryptic Soy Agar |
| 5 | Burkholderia cepacia | 25608 | 15 and 30 sec | Tryptic Soy Agar |
| 6 | Escherichla coli | 11775 | 15 and 30 sec | Tryptic Soy Agar |
| 7 | Escherichia coli | 25922 | 15 and 30 sec | Tryptic Soy Agar |
| 8 | Klebsiella pneumoniae | 13883 | 15 and 30 sec | Tryptic Soy Agar |
| 9 | Klebsiella pneumoniae | 27736 | 15 and 30 sec | Tryptic Soy Agar |
| 10 | Pseudomonas aeruginosa | 15442 | 15 and 30 sec | Tryptic Soy Agar |
| 11 | Pseudomonas aeruginosa | 27853 | 1S and 30 sec | Tryptic Soy Agar |
| 12 | Serratia marcescens | 8100 | 15 and 30 sec | Tryptic Soy Agar |
| 13 | Serratia marcescens | 14756 | 15 and 30 sec | Tryptic Soy Agar |
| 14 | Campylobacter jejuni | 33291 | 15 and 30 sec | Blood Agar 5% Sheep blood |
| 15 | Campylobacter jejuni | 49943 | 15 and 30 sec | Blood Agar 5% Sheep blood |
| 16 | Salmonella enterica serovar | 13076 | 15 and 30 sec | Tryptic Soy Agar |
| | enteritidis | | | |
| 17 | Salmonella enterica serovar | 14028 | 15 and 30 sec | Tryptic Soy Agar |
| | typhimurium | | | |
| 18 | Shigella sonnei | 9290 | 15 and 30 sec | Tryptic Soy Agar |
| 19 | Shigella sonnei | 25931 | 15 and 30 sec | Tryptic Soy Agar |
| 20 | Enterococcus faecalis | 19433 | 15 and 30 sec | Brain Heart Infusion Agar |
| 21 | Enterococcus faecalis | 29212 | 15 and 30 sec | Brain Heart Infusion Agar |
| 22 | Staphylocaccus aureus | 6538 | 15 and 30 sec | Tryptic Soy Agar |
| 23 | Staphylocaccus aureus | 29213 | 15 and 30 sec | Tryptic Soy Agar |
| 24 | Methicillin-resistant | 33591 | 15 and 30 sec | Tryptic Soy Agar |
| | Staphylococcus aureus | | | |
| 25 | Methicillin-resistant | 33592 | 15 and 30 sec | Tryptic Soy Agar |
| | Staphylococcus aureus | | | |
| 26 | Streptococcus pyogenes | 14289 | 15 and 30 sec | Blood Agar 5% Sheep blood |
| 27 | Streptococcus pyogenes | 19615 | 15 and 30 sec | Blood Agar S% Sheep blood |
| 28 | Listeria manocyrogenes | 7644 | 15 and 30 sec | in Heart Infusion Agar |
| 29 | Listeria monocyrogenes | 19115 | 15 and 30 sec | Brain Heart Infusion Agar |
| 30 | Streptococcus pneumoniae | 6303 | 15 and 30 sec | Blood Agar 5% Sheep blood |
| 31 | Streptococcus pneumoniae | 49619 | 15 and 30 sec | Blood Agar 5% Sheep blood |

Test samples of the formulation were tested neat, or non-diluted. The testing was conducted at room temperature (20-25° C.) in three replicates with each replicated plated in duplicate. Testing was performed on 25 mL or gram aliquots of the formulation. Three containers of the formulation were tested for each test organism.

Aerobic organisms were prepared by transferring twice (once every 18-24 hours) on appropriate agar as required by the organism and incubating at approximately 36-38° C. for 24 hours minimum. The second transfer was made onto appropriate agar plate or slant and inoculum prepared by washing the plate or slant with 5-10 mL of sterile PBS or sterile saline.

Anaerobic organisms were prepared by transferring twice (once every 2-3 days) on the appropriate anaerobic medium as required by the organism and incubating at approximately 36-38° C. under anaerobic conditions. The second transfer was made onto the same media previously used for the organism and inoculum prepared by washing the plate with 5-10 mL of sterile PBS or sterile saline.

To enumerate inoculum used for time kill procedures, approximately 108 CFU/mL inoculum was plated in triplicate for each organism by either pour plate or spread plate technique. Plates were incubated at 36-38° C. for 24 hours minimum for aerobes and 2-3 days minimum under anaerobic conditions for anaerobes. Colonies were then counted and recorded as CFU/mL.

Time kill procedures were performed in triplicate. The procedures were initiated by inoculating 25 grams or mL of the test formulation or a 25 mL of a blank containing sterile DI water with 0.25 mL of test organism in suspension, resulting in approximately 10⁶ CFU/mL. At 15 and 30 seconds, 1 mL of test formulation/organism mixture was transferred to 9 mL Dey-Engley neutralizing broth (D/E neutralizing broth), which was serially diluted into 10⁻¹, 10⁻², 10⁻³, and 10⁻⁴ dilutions. The serial dilutions were plated in duplicate by either pour plate or spread plate technique using the appropriate agar for the test organism. Plates were incubated at 36-38° C. for 24 hours minimum for aerobes and 2-3 days minimum under anaerobic conditions for anaerobes. Plates containing 30-300 colonies per plate were used for calculations where possible. The number of surviving organisms after 15 and 30 seconds was determined by averaging the plate counts, correcting for dilution, and log transforming the corrected value. This log transformed value is expressed as the result. Reduction in counts after exposure to the test formulation compared to the blank indicate efficacy of the formulation.

Results from the time kill procedures are reported in the tables below as the number of surviving organisms over time.

TABLE 3
Results for Time Kill Assay at 15 and 30 seconds Contact Time against
Haemophilus influenzae ATCC 33391

| CPTC Test | | | |
|---|---|---|---|
| Article ID# | Control Counts | M20-3441.01 | |
| Contact Time | Initial Count (Time 0) | 15 seconds | 30 seconds |
| Haemophilus influenzae | | | |
| ATCC 33391 | | | |
| Replicate | | | |
| A | 6.7 × 10⁵ | <100 | <100 |
| B | 6.4 × 10⁵ | <100 | <100 |
| C | 6.9 × 10⁵ | <100 | <100 |
| Average Count | 6.7 × 10⁵ | <100 | <100 |
| Log₁₀ Average | 5.82 | <2.00 | <2.00 |
| Log₁₀ Reduction | NA | >3.82 | >3.82 |
| % Reduction | NA | >99.9 | >99.9 |

TABLE 4
Results for Time Kill Assay at 15 and 30 seconds Contact Time
against Bacteroides fragilis ATCC 25285

| CPTC Test | | | |
|---|---|---|---|
| Article ID# | Control Counts | M20-3441.01 | |
| Contact Time | Initial Count (Time 0) | 15 seconds | 30 seconds |
| Bacteroides fragilis | | | |
| ATCC 25825 | | | |
| Replicate | | | |
| A | 1.7 × 10⁶ | <100 | <100 |
| B | 1.4 × 10⁶ | <100 | <100 |
| C | 1.5 × 10⁶ | <100 | <100 |
| Average Count | 1.5 × 10⁶ | <100 | <100 |
| Log₁₀ Average | 6.17 | <2.00 | <2.00 |
| Log₁₀ Reduction | NA | >4.17 | >4.17 |
| % Reduction | NA | >99.99 | >99.99 |

TABLE 5
Results for Time Kill Assay at 15 and 30 seconds Contact Time
against Enterobacter cloacae ATCC 13047

| CPTC Test | | | |
|---|---|---|---|
| Article ID# | Control Counts | M20-3441.01 | |
| Contact Time | Initial Count (Time 0) | 15 seconds | 30 seconds |
| Enterobacter cloacae | | | |
| ATCC 13047 | | | |
| Replicate | | | |
| A | 1.9 × 10⁶ | <10 | <10 |
| B | 1.9 × 10⁶ | <10 | <10 |
| C | 1.8 × 10⁶ | <10 | <10 |
| Average Count | 1.9 × 10⁶ | <10 | <10 |
| Log₁₀ Average | 6.27 | <1.00 | <1.00 |
| Log₁₀ Reduction | NA | >5.27 | >5.27 |
| % Reduction | NA | >99.999 | >99.999 |

TABLE 6
Results for Time Kill Assay at 15 and 30 seconds Contact Time against
Burkholderia cepacia ATCC 25416

| CPTC Test | | | |
|---|---|---|---|
| Article ID# | Control Counts | M20-3441.01 | |
| Contact Time | Initial Count (Time 0) | 15 seconds | 30 seconds |
| Burkholderia cepacia | | | |
| ATCC 25416 | | | |
| Replicate | | | |
| A | 1.6 × 10⁶ | <10 | <10 |
| B | 1.6 × 10⁶ | <10 | <10 |
| C | 1.6 × 10⁶ | <10 | <10 |
| Average Count | 1.6 × 10⁶ | <10 | <10 |
| Log₁₀ Average | 6.20 | <1.00 | <1.00 |
| Log₁₀ Reduction | NA | >5.20 | >5.20 |
| % Reduction | NA | >99.999 | >99.999 |

TABLE 7
Results for Time Kill Assay at 15 and 30 seconds Contact Time
against Burkholderia cepacia ATCC 25608

| CPTC Test | | | |
|---|---|---|---|
| Article 1D# | Control Counts | M20-3441.01 | |
| Contact Time | Initial Count (Time 0) | 15 seconds | 30 seconds |
| Burkholderia | | | |
| cepocia | | | |
| ATCC 25608 | | | |
| Replicate | | | |
| A | 1.2 × 10⁶ | <10 | <10 |
| B | 1.2 × 10⁶ | <10 | <10 |
| C | 1.2 × 10⁶ | <10 | <10 |
| Average Count | 1.2 × 10⁶ | <10 | <10 |
| Log₁₀ Average | 6.07 | <1.00 | <1.00 |
| Log₁₀ Reduction | NA | >5.07 | >5.07 |
| % Reduction | NA | >99.999 | >99.999 |

TABLE 8
Results for Time Kill Assay at 15 and 30 seconds Contact Time
against Escherichia coli ATCC 11775

| CPTC Test | | | |
|---|---|---|---|
| Article ID# | Control Counts | M20-3441.01 | |
| Contact Time | Initial Count (Time 0) | 15 seconds | 30 seconds |
| Escherichia Coli | | | |
| ATCC 11775 | | | |
| Replicate | | | |
| A | 1.4 × 10⁶ | <10 | <10 |
| B | 1.4 × 10⁶ | <10 | <10 |
| C | 1.4 × 10⁶ | <10 | <10 |
| Average Count | 1.4 × 10⁶ | <10 | <10 |
| Log₁₀ Average | 6.14 | <1.00 | <1.00 |
| Log₁₀ Reduction | NA | >5.14 | >5.14 |
| % Reduction | NA | >99.999 | >99.999 |

TABLE 9
Results for Time Kill Assay at 15 and 30 seconds Contact Time
against Escherichia coli ATCC 25922

| CPTC Test | | | |
|---|---|---|---|
| Article ID# | Control Counts | M20-3441.01 | |
| Contact Time | Initial Count (Time 0) | 15 seconds | 30 seconds |
| Escherichia coli | | | |
| ATCC 25922 | | | |
| Replicate | | | |
| A | 1.1 × 10⁶ | <10 | <10 |
| B | 1.0 × 10⁶ | <10 | <10 |
| C | 1.0 × 10⁶ | <10 | <10 |
| Average Count | 1.0 × 10⁶ | <10 | <10 |
| Log₁₀ Average | 6.00 | <1.00 | <1.0 |
| Log₁₀ Reduction | NA | >5.00 | >5.00 |
| % Reduction | NA | >99.999 | >99.999 |

TABLE 10
Results for Time Kill Assay at 15 and 30 seconds Contact Time
against Klebsiella pneumoniae ATCC 13883

| CPTC Test | | | |
|---|---|---|---|
| Article ID # | Control Counts | M20-3441.01 | |
| Contact Time | Initial Count (Time 0) | 15 seconds | 30 seconds |
| Kiebsiella pneumoniae | | | |
| ATCC 13883 | | | |
| Replicate | | | |
| A | 1.5 × 10⁶ | <10 | <10 |
| B | 1.6 × 10⁶ | <10 | <10 |
| C | 1.5 × 10⁶ | <10 | <10 |
| Average Count | 1.5 × 10⁶ | <10 | <10 |
| Log₁₀ Average | 6.17 | <1.00 | <1.00 |
| Log₁₀ Reduction | NA | >5.17 | >5.17 |
| % Reduction | NA | >99.999 | >99.999 |

TABLE 11
Results for Time Kill Assay at 15 and 30 seconds Contact Time
against Klebsiella pneumoniae ATCC 27736

| CPTC Test | | | |
|---|---|---|---|
| Article ID# | Control Counts | M20-3441.01 | |
| Contact Time | initial Count (Time 0) | 15 seconds | 30 seconds |
| Klebsiella pneumoniae | | | |
| ATCC 27736 | | | |
| Replicate | | | |
| A | 1.2 × 10⁶ | <10 | <10 |
| B | 1.1 × 10⁶ | <10 | <10 |
| C | 1.1 × 10⁶ | <10 | <10 |
| Average Count | 1.1 × 10⁶ | <10 | <10 |
| Log₁₀ Average | 6.04 | <1.00 | <1.00 |
| Log₁₀ Reduction | NA | >5.04 | >5.04 |
| % Reduction | NA | >99.999 | >99.999 |
| I | | | |

TABLE 12
Results for Time Kill Assay at 15 and 30 seconds Contact Time
against Pseudomonas aeruginosa ATCC 15442

| CPTC Test | | | |
|---|---|---|---|
| Article ID# | Control Counts | M20-3441.01 | |
| Contact Time | Initial Count (time 0) | 15 seconds | 30 seconds |
| Pseudamonas aeruginosa | | | |
| ATCC 15442 | | | |
| Replicate | | | |
| A | 1.8 × 10⁶ | <10 | <10 |
| B | 1.8 × 10⁶ | <10 | <10 |
| C | 1.7 × 10⁶ | <10 | <10 |
| Average Count | 1.8 × 10⁶ | <10 | <10 |
| Log₁₀ Average | 6.25 | <1.00 | <1.00 |
| Log₁₀ Reduction | NA | >5.25 | >5.25 |
| % Reduction | NA | >99.999 | >99.999 |

TABLE 13
Results for Time Kill Assay at 15 and 30 seconds Contact Time
against Pseudomonas aeruginosa ATCC 27853

| CPTC Test | | | |
|---|---|---|---|
| Article ID# | Control Counts | M20-3441.01 | |
| Contact Time | Initial Count (Time 0) | 15 seconds | 30 seconds |
| Pseudomonas aeruginasa | | | |
| ATCC 27853 | | | |
| Replicate | | | |
| A | 8.7 × 10⁵ | <10 | <10 |
| B | 8.9 × 10⁵ | <10 | <10 |
| C | 9.3 × 10⁵ | <10 | <10 |
| Average Count | 9.0 × 10⁵ | <10 | <10 |
| Log₁₀ Average | 5.95 | <1.00 | <1.00 |
| Log₁₀ Reduction | NA | >4.95 | >4.95 |
| % Reduction | NA | >99.99 | >99.99 |

TABLE 14
Results for Time Kill Assay at 15 and 30 seconds Contact Time
against Serratia marcescens ATCC 8100

| CPTC Test | | | |
|---|---|---|---|
| Article ID# | Control Counts | M20-3441.01 | |
| Contact Time | Initial Count (Time 0) | 15 seconds | 30 seconds |
| Serratia marcescens | | | |
| ATCC 8100 | | | |
| Replicate | | | |
| A | 1.6 × 10⁶ | <10 | <10 |
| B | 1.6 × 10⁶ | <10 | <10 |
| C | 1.5 × 10⁶ | <10 | <10 |
| Average Count | 1.6 × 10⁶ | <10 | <10 |
| Log₁₀ Average | 5.20 | <1.00 | <1.00 |
| Log₁₀ Reduction | NA | >5.20 | >5.20 |
| % Reduction | NA | >99.999 | >99.999 |

TABLE 15
Results for Time Kill Assay at 15 and 30 seconds Contact Time
against Serratia marcescens ATCC 14756

| CPTC Test | | | |
|---|---|---|---|
| Article ID# | Control Counts | M20-3441.01 | |
| Contact Time | Initial Count (Time 0) | 5 seconds | 30 seconds |
| Serratia marrescens | | | |
| ATCC 14756 | | | |
| Replicate | | | |
| A | 1.8 × 10⁶ | <10 | <10 |
| B | 1.7 × 10⁶ | <10 | <10 |
| C | 1.8 × 10⁶ | <10 | <10 |
| Average Count | 1.8 × 10⁶ | <10 | <10 |
| Log₁₀ Average | 6.25 | <1.00 | <1.00 |
| Log₁₀ Reduction | NA | >5.25 | >5.25 |
| % Reduction | NA | >99.999 | >99.999 |

TABLE 16
Results for Time Kill Assay at 15 and 30 seconds Contact Time
against Campylobacter jejuni ATCC 33291

| CPTC Test | | | |
|---|---|---|---|
| Article ID# | Control Counts | M20-3441.01 | |
| Contact Time | Initial Count (Time 0) | 15 seconds | 30 seconds |
| Campylobacter jejuni | | | |
| ATCC 33291 | | | |
| Replicate | | | |
| A | 1.9 × 10⁶ | <100 | <100 |
| B | 1.8 × 10⁶ | <100 | <100 |
| C | 1.8 × 10⁶ | <100 | <100 |
| Average Count | 1.8 × 10⁵ | <100 | <100 |
| Log₁₀ Average | 6.25 | <2.00 | <2.00 |
| Log₁₀ Reduction | NA | >4.25 | >4.25 |
| % Reduction | NA | >99.99 | >99.99 |

TABLE 17
Results for Time Kill Assay at 15 and 30 seconds Contact Time
against Campylobacter jejuni ATCC 49943

| CPTC Test | | | |
|---|---|---|---|
| Article ID# | Control Counts | M20-3441.01 | |
| Contact Time | Initial Count (Time 0) | 15 seconds | 30 seconds |
| Compylobacter jejuni | | | |
| ATCC 49943 | | | |
| Replicate | | | |
| A | 1.6 × 10⁶ | <100 | <100 |
| B | 1.5 × 10⁶ | <100 | <100 |
| C | 1.6 × 10⁶ | <100 | <100 |
| Average Count | 1.6 × 10⁶ | <100 | <100 |
| Log₁₀Average | 6.20 | <2.00 | <2.00 |
| Log₁₀Reduction | NA | >4.20 | >4.20 |
| % Reduction | NA | >99.99 | >99.99 |

TABLE 18
Results for Time Kill Assay at 15 and 30 seconds Contact Time
against Salmonella enterica serovar enteritidis ATCC 13076

| CPTC Test | | | |
|---|---|---|---|
| Article ID# | Control Counts | M20-3441.01 | |
| Contact Time | Initial Count (Time 0) | 15 seconds | 30 seconds |
| Salmonella enterica | | | |
| serovar enteritidis | | | |
| ATCC 13076 | | | |
| Replicate | | | |
| A | 1.7 × 10⁶ | <10 | <10 |
| B | 1.9 × 10⁶ | <10 | <10 |
| C | 1.5 × 10⁶ | <10 | <10 |
| Average Count | 1.7 × 10⁶ | <10 | <10 |
| Log₁₀ Average | 6.23 | <1.00 | <1.00 |
| Log₁₀ Reduction | NA | >5.23 | >5.23 |
| % Reduction | NA | >99.999 | >99.999 |

TABLE 19
Results for Time Kill Assay at 15 and 30 seconds Contact Time
against Salmonella enterica serovar typhimurium ATCC 14028

| CPTC Test | | | |
|---|---|---|---|
| Article ID# | Control Counts | M20-3441.01 | |
| Contact Time | Initial Count (Time 0) | 15 seconds | 30 seconds |
| Salmonella enterica | | | |
| serovar typhimurium | | | |
| ATCC 14028 | | | |
| Replicate | | | |
| A | 1.3 × 10⁶ | <10 | <10 |
| B | 1.3 × 10⁶ | <10 | <10 |
| C | 1.3 × 10⁶ | <10 | <10 |
| Average Count | 1.3 × 10⁶ | <10 | <10 |
| Log₁₀ Average | 6.11 | <1.00 | <1.00 |
| Log₁₀ Reduction | NA | >5.11 | >5.11 |
| % Reduction | NA | >99.999 | >99.999 |

TABLE 20
Results for Time Kill Assay at 15 and 30 seconds Contact Time
against Shigella sonnei ATCC 9290

| CPTC Test | | | |
|---|---|---|---|
| Article ID# | Control Counts | M20-3441.01 | |
| Contact Time | Initial Count (Time 0) | 15 seconds | 30 seconds |
| Shigetia sonnei | | | |
| ATCC 9290 | | | |
| Replicate | | | |
| A | 1.1 × 10⁶ | <10 | <10 |
| B | 1.2 × 10⁶ | <10 | <10 |
| C | 1.2 × 10⁶ | <10 | <10 |
| Average Count | 1.2 × 10⁶ | <10 | <10 |
| Log₁₀ Average | 6.07 | <1.00 | <1.00 |
| Log₁₀ Reduction | NA | >5.07 | >5.07 |
| % Reduction | NA | >99.999 | >99.999 |

TABLE 21
Results for Time Kill Assay at 15 and 30 seconds Contact Time
against Shigella sonnei ATCC 25931

| CPTC Test | | | |
|---|---|---|---|
| Article ID# | Control Counts | M20-3441.01 | |
| Contact Time | Initial Count (Time 0) | 15 seconds | 30 seconds |
| Shigella somei | | | |
| ATCC 25931 | | | |
| Replicate | | | |
| A | 1.0 × 10⁶ | <10 | <10 |
| B | 1.0 × 10⁶ | <10 | <10 |
| C | 9.9 × 10⁶ | <10 | <10 |
| Average Count | 1.0 × 10⁶ | <10 | <10 |
| Log₁₀ Average | 6.00 | <1.00 | <1.00 |
| Log₁₀ Reduction | NA | >5.00 | >5.00 |
| % Reduction | NA | >99.999 | >99.999 |

TABLE 22
Results for Time Kill Assay at 15 and 30 seconds Contact Time against
Enterococcus faecalis ATCC 19433

| CPTC Test | | | |
|---|---|---|---|
| Article ID # | Control Counts | M20-3441.01 | |
| Contact Time | Initial Count (Time 0) | 15 seconds | 30 seconds |
| Enterococcus faecalis | | | |
| ATCC 19433 | | | |
| Replicate | | | |
| A | 1.6 × 10⁶ | <10 | <10 |
| B | 1.6 × 10⁶ | <10 | <10 |
| C | 1.6 × 10⁶ | <10 | <10 |
| Average Count | 1.6 × 10⁶ | <10 | <10 |
| Log₁₀ Average | 6.20 | <1.00 | <1.00 |
| Log₁₀ Reduction | NA | >5.20 | >5.20 |
| % Reduction | NA | >99.999 | >99.999 |

TABLE 23
Results for Time Kill Assay at 15 and 30 seconds Contact Time against
ATCC 29212 Enterococcus faecalis

| CRTC Test | | | |
|---|---|---|---|
| Article ID # | Control Counts | M20-3441.01 | |
| Contact Time | Initial Count (Time 0) | 15 seconds | 30 seconds |
| Enterococcus faecalis | | | |
| ATCC 29212 | | | |
| Replicate | | | |
| A | 1.5 × 10⁶ | <10 | <10 |
| B | 1.5 × 10⁶ | <10 | <10 |
| C | 1.5 × 10⁶ | <10 | <10 |
| Average Count | 1.5 × 10⁶ | <10 | <10 |
| Log₁₀ Average | 6.17 | <1.00 | <1.00 |
| Log₁₀ Reduction | NA | >5.17 | >5.17 |
| % Reduction | NA | >99.999 | >99.999 |

TABLE 24
Results for Time Kill Assay at 15 and 30 seconds Contact Time against
Staphylococcus aureus ATCC 6538

| CPTC Test | | | |
|---|---|---|---|
| Article ID # | Control Counts | M20-3441.01 | |
| Contact Time | Initial Count (Time 0) | 15 seconds | 30 seconds |
| Staphylococcus aureus | | | |
| ATCC 6538 | | | |
| Replicate | | | |
| A | 1.4 x 10⁶ | <10 | <10 |
| B | 1.4 x 10⁶ | <10 | <10 |
| C | 1.3 x 10⁶ | <10 | <10 |
| Average Count | 1.4 x 10⁶ | <10 | <10 |
| Log₁₀ Average | 6.14 | <1.00 | <1.00 |
| Log₁₀ Reduction | NA | >5.14 | >5.14 |
| % Reduction | NA | >99.999 | >99.999 |

TABLE 25
Results for Time Kill Assay at 15 and 30 seconds Contact Time against
Staphylococcus aureus ATCC 29213

| CPTC Test | | | |
|---|---|---|---|
| Article ID # | Control Counts | M20-3441.01 | |
| Contact Time | Initial Count (Time 0) | 15 seconds | 30 seconds |
| Staphylococcus aureus | | | |
| ATCC 29213 | | | |
| Replicate | | | |
| A | 1.6 x 10⁶ | <10 | <10 |
| B | 1.4 x 10⁶ | <10 | <10 |
| C | 1.4 x 10⁶ | <10 | <10 |
| Average Count | 1.5 x 10⁶ | <10 | <10 |
| Log₁₀ Average | 6.17 | <1.00 | <1.00 |
| Log₁₀ Reduction | NA | >5.17 | >5.17 |
| % Reduction | NA | >99.999 | >99.999 |

TABLE 26
Results for Time Kill Assay at 15 and 30 seconds Contact Time against
Methicillin-resistant Staphylococcus aureus ATCC 33591

| CPTC Test | | | |
|---|---|---|---|
| Article ID # | Control Counts | M20-3441.01 | |
| Contact Time | Intial Count (Time 0) | 15 seconds | 30 seconds |
| Methicillin-resistant | | | |
| Staphylococcus aureus | | | |
| ATCC 33591 | | | |
| Replicate | | | |
| A | 1.3 x 10⁶ | <10 | <10 |
| B | 1.2 x 10⁶ | <10 | <10 |
| C | 1.3 x 10⁶ | <10 | <10 |
| Average Count | 1.3 x 10⁶ | <10 | <10 |
| Log₁₀ Average | 6.11 | <1.00 | <1.00 |
| Log₁₀ Reduction | NA | >5.11 | >5.11 |
| % Reduction | NA | >99.999 | >99.999 |

TABLE 27
Results for Time Kill Assay at 15 and 30 seconds Contact Time against
Methicillin-resistant Staphylococcus aureus ATCC 33592

| CPTC Test | | | |
|---|---|---|---|
| Article ID # | Control Counts | M20-3441.01 | |
| Contact Time | Initial Count (Time 0) | 15 seconds | 30 seconds |
| Methicillin-resistant | | | |
| Staphyiococcus mucus | | | |
| ATCC 33592 | | | |
| Replicate | | | |
| A | 1.4 x 10⁶ | <10 | <10 |
| B | 1.5 x 10⁶ | <10 | <10 |
| C | 1.4 x 10⁶ | <10 | <10 |
| Average Count | 1.4 x 10⁶ | <10 | <10 |
| Log₁₀ Average | 6.14 | <1.00 | <1.00 |
| Log₁₀ Reduction | NA | >5.14 | >5.14 |
| % Reduction | NA | >99.999 | >99.999 |

TABLE 28
Results for Time Kill Assay at 15 and 30 seconds Contact Time against
Streptococcus pyogenes ATCC 14289

| CPTC Test | | | |
|---|---|---|---|
| Article ID # | Control Counts | M20-3441.01 | |
| Contact Time | Initial Count (Time 0) | 15 seconds | 30 seconds |
| Streptococcus pyogenes | | | |
| ATCC 14289 | | | |
| Replicate | | | |
| A | 1.6 x 10⁶ | <10 | <10 |
| B | 1.6 x 10⁶ | <10 | <10 |
| C | 1.6 x 10⁶ | <10 | <10 |
| Average Count | 1.6 x 10⁶ | <10 | <10 |
| Log₁₀ Average | 6.20 | <1.00 | <1.00 |
| Log₁₀ Reduction | NA | >4.20 | >4.20 |
| % Reduction | NA T | >99.99 | >99.99 |

TABLE 29
Results for Time Kill Assay at 15 and 30 seconds Contact Time against
Streptococcus pyogenes ATCC 19615

| CPTC Test | | | |
|---|---|---|---|
| Article ID # | Control Counts | M20-3441.01 | |
| Contact Time | Initial Count (Time 0) | 15 seconds | 30 seconds |
| Streptococcus pyogenes | | | |
| ATCC 19615 | | | |
| Replicate | | | |
| A | 1.0 x 10⁶ | <10 | <10 |
| B | 9.8 x 10⁶ | <10 | <10 |
| C | 1.0 x 10⁶ | <10 | <10 |
| Average Count | 1.0 x 10⁶ | <10 | <10 |
| Log₁₀ Average | 6.00 | <1.00 | <1.00 |
| Log₁₀ Reduction | NA | >4.00 | >4.00 |
| % Reduction | NA | >99.99 | >99.99 |

TABLE 30
Results for Time Kill Assay at 15 and 30 seconds Contact Time
against Listeria monocytogenes ATCC 7644

| CPTC Test | | | |
|---|---|---|---|
| Article ID # | Control Counts | M20-3441.01 | |
| Contact Time | Initial Count (Time 0) | 15 seconds | 30 seconds |
| Listeria monocytogenes | | | |
| ATCC 7644 | | | |
| Replicate | | | |
| A | 1.9 x 10⁶ | <10 | <10 |
| B | 1.8 x 10⁶ | <10 | <10 |
| C | 1.9 x 10⁶ | <10 | <10 |
| Average Count | 1.9 x 10⁶ | <10 | <10 |
| Log₁₀ Average | 6.27 | <1.00 | <1.00 |
| Log₁₀ Reduction | NA | >5.27 | >5.27 |
| % Reduction | NA | >99.999 | >99.999 |

TABLE 31
Results for Time Kill Assay at 15 and 30 seconds Contact Time
against Listeria monocytogenes ATCC 19115

| CPTC Test | | | |
|---|---|---|---|
| Article ID # | Control Counts | M20-3441.01 | |
| Contact Time | initial Count (Time 0) | 5 seconds | 30 seconds |
| Listeria monocytogenes | | | |
| ATCC 19115 | | | |
| Replicate | | | |
| A | 1.9 x 10⁶ | <10 | <10 |
| B | 1.9 x 10⁶ | <10 | <10 |
| C | 1.9 x 10⁶ | <10 | <10 |
| Average Count | 1.9 x 10⁶ | <10 | <10 |
| Log₁₀ Average | 6.27 | <1.00 | <1.00 |
| Log₁₀ Reduction | NA | >5.27 | >5.27 |
| % Reduction | NA | >99.999 | >99.999 |

TABLE 32
Results for Time Kill Assay at 15 and 30 seconds Contact Time against
Streptococcus pneumoniae ATCC 6303

| CPTC Test | | | |
|---|---|---|---|
| Article ID # | Control Counts | M20-3441.01 | |
| Contact Time | Initial Count (Time 0) | 15 seconds | 30 seconds |
| Streptococcus pneumoniae | | | |
| ATCC 6303 | | | |
| Replicate | | | |
| A | 9.6 x 10⁶ | <10 | <10 |
| B | 1.0 x 10⁶ | <10 | <10 |
| C | 1.0 x 10⁶ | <10 | <10 |
| Average Count | 9.9 x 10⁶ | <10 | <10 |
| Log₁₀ Average | 5.99 | <1.00 | <1.00 |
| Log₁₀ Reduction | NA | >3.99 | >3.99 |
| % Reduction | NA | >99.9 | >99.9 |

TABLE 33
Results for Time Kill Assay at 15 and 30 seconds Contact Time against
Streptococcus pneumoniae ATCC 49619

| CPTC Test | | | |
|---|---|---|---|
| Article ID # | Control Counts | M20-3441.01 | |
| Contact Time | Initial Count (Time 0) | 15 seconds | 30 seconds |
| Streptococcus pneumoniae | | | |
| ATCC 49619 | | | |
| Replicate | | | |
| A | 1.3 x 10⁶ | <10 | <10 |
| B | 1.6 x 10⁶ | <10 | <10 |
| C | 1.7 x 10⁶ | <10 | <10 |
| Average Count | 1.7 x 10⁶ | <10 | <10 |
| Log₁₀ Average | 6.23 | <1.00 | <1.00 |
| Log₁₀ Reduction | NA | >4.23 | >4.23 |
| % Reduction | NA | >99.99 | >99.99 |

TABLE 34
Summary of Log Reductions and
Percent Reductions for Test Formulation

| | ATCC | Log Reduction | | % Reduction | |
|---|---|---|---|---|---|
| Test Organism | Number | 15 seconds | 30 seconds | 15 seconds | 30 seconds |
| Haemophilus | 33391 | >3.82 | >3.82 | >99.9 | >99.9 |
| infuenzae | | | | | |
| Bacteroides | 25285 | >4.17 | >4.17 | >99.99 | >99.99 |
| fragilis | | | | | |
| Enterobacter | 13047 | >5.27 | >5.27 | >99.999 | >99.999 |
| cloacae | | | | | |
| Burkholderia | 25416 | >5.20 | >5.20 | >99.999 | >99.999 |
| cepacia | | | | | |
| Burkholderia | 25608 | >5.07 | >5.07 | >99.999 | >99.999 |
| cepacio | | | | | |
| Escherichia coli | 11775 | >5.14 | >5.14 | >99.999 | >99.999 |
| Escherichia coli | 25927 | >5.00 | >5.00 | >99.999 | >99.999 |
| Kiebsiella | 13883 | >5.17 | >5.17 | >99.999 | >99.999 |
| pneumoniae | | | | | |
| Kiebsiella | 27736 | >5.04 | >5.04 | >99.999 | >99.999 |
| pneumoniae | | | | | |
| Pseudomonas | 15442 | >5.75 | >5.75 | >99.999 | >99.999 |
| aeruginosa | | | | | |
| Pseudomonas | 27853 | >4.95 | >4.95 | >99.99 | >99.99 |
| aeruginosa | | | | | |
| Serratia | 8100 | >5.20 | >5.20 | >99.999 | >99.999 |
| marcescens | | | | | |
| Serratia | 14756 | >5.25 | >5.25 | >99.999 | >99.999 |
| marcescens | | | | | |
| Campylobacter | 33291 | >4.25 | >4.25 | >99.99 | >99.99 |
| jejuni | | | | | |
| Campylobacter | 49943 | >4.20 | >4.20 | >99.99 | >99.99 |
| jejuni | | | | | |
| Salmonella | 13076 | >5.23 | >5.23 | >99.999 | >99.999 |
| enterica | | | | | |
| seravar | | | | | |
| enteritidis | | | | | |
| Salmonella | 14028 | >5.11 | >5.11 | >99.999 | >99.999 |
| enterica | | | | | |
| seravar | | | | | |
| typhimurium | | | | | |
| Shigella sonnei | 9290 | >5.07 | >5.07 | >99.999 | >99.999 |
| Shigella sonnei | 25931 | >5.00 | >5.00 | >99.999 | >99.999 |
| Enterococcus | 19433 | >5.20 | >5.20 | >99.999 | >99.999 |
| faecalis | | | | | |
| Enterococcus | 29212 | >5.17 | >5.17 | >99.999 | >99.999 |
| faecalis | | | | | |
| Staphylococcus | 6538 | >5.14 | >5.14 | >99.999 | >99.999 |
| aureus | | | | | |
| Staphylococcus | 29213 | >5.17 | >5.17 | >99.999 | >99.999 |
| aureus | | | | | |
| Methivillin- | 33591 | >5.11 | >5.11 | >99.999 | >99.999 |
| resistant | | | | | |
| Staphylococcus | | | | | |
| aureus | | | | | |
| Methivillin- | 33592 | >5.14 | >5.14 | >99.999 | >99.999 |
| resistant | | | | | |
| Staphylococcus | | | | | |
| aureus | | | | | |
| Streptococcus | 142895 | >4.00 | >4.00 | >99.99 | >99.99 |
| pyogenes | | | | | |
| Streptococcus | 7644 | >5.27 | >5.27 | >99.999 | >99.999 |
| pyogenes | | | | | |
| Listeria | 19115 | >5.21 | >5.21 | >99.999 | >99.9 |
| monocytogenes | | | | | |
| Streptococcus | 6303 | >3.99 | >3.99 | | |
| pneumoniae | | | | | |
| Streptococcus | 49619 | >4.23 | >4.23 | >99.99 | >99.99 |
| pneumoniae | | | | | |

The results of the in vitro time kill testing for the test formulation against 31 test organisms showed antimicrobial activity with log reductions (percent reductions) ranging from the lowest log reduction of >3.82 logs (>99.9%) to the highest log reduction of >5.27 logs (99.999%).

### Example 3

### Neutralization Effectiveness

The presence of the active preservative phenoxyethanol in the tested formulation carried over from the challenged test samples in the plating diluent and recovery medium during sampling may inhibit viable microorganisms and result in false-negative readings. Therefore, neutralizing agents are incorporated into the plating diluent and/or recovery medium to inactivate the preservative and permit accurate enumeration of microbial content. The choice of neutralizer is based on the type of preservative system. For example, a lecithin-based neutralizer is used for paraben preservative systems, while a thiosulfate-based neutralizer is used for halogen-based preservative systems. The neutralizer should be evaluated prior to or concurrently with testing to determine if the preservative system is effectively neutralized.

Testing for neutralizer effectiveness on the phenoxyethanol of the test formulation was therefore performed against the 31 microbial organisms listed in Table 2 above. Tests were done in triplicate with each replicate plated in duplicate for 15 and 30 second time points, with all appropriate controls.

Aerobic organisms were prepared by transferring twice (once every 18-24 hours) on appropriate agar as required by the organism and incubating at approximately 36-38° C. for 24 hours minimum. The second transfer was made onto appropriate agar plate or slant and inoculum prepared by washing the plate or slant with 5-10 mL of sterile saline.

Anaerobic organisms were prepared by transferring twice (once every 2-3 days) on the appropriate anaerobic medium as required by the organism and incubating at approximately 36-38° C. under anaerobic conditions. The second transfer was made onto the same media previously used for the organism and inoculum prepared by washing the plate with 5-10 mL of sterile saline.

The concentration of the test organism (aerobic or anaerobic) was adjusted spectrophotometrically in sterile saline to a concentration of approximately 10⁸ CFU/mL. The adjusted organism was serially diluted to approximately 10³-10⁴ CFU/mL.

To test neutralization of the phenoxyethanol preservative system of the test formulation, one gram of the test formulation was inoculated into 9 mL of Dey-Engley (D/E) neutralizing both for each individual test organism to achieve a 1:10 and 1:100 dilution. D/E broth is capable of neutralizing a broad spectrum of antiseptic and disinfectant chemicals including quaternary ammonium compounds, phenolics, iodine, chlorine preparations, mercurials, formaldehyde and glutaraldehyde. It contains various neutralizing agents: lecithin, Tween®, sodium thiosulfate, and sodium bisulfite. Lecithin neutralizes quaternary ammonia compounds while phenolic disinfectants, like phenoxyethanol, and hexachlorophene are neutralized by Tween®. Together, lecithin and Tween® neutralize ethanol. Sodium thiosulfate neutralizes iodine and chlorine, and sodium bisulfite neutralizes formaldehyde and gluteraldehyde. D/E Neutralizing media therefore effectively neutralizes the inhibitory effects of phenoxyethanol carryover, allowing differentiation between bacteriostasis due to the phenoxyethanol and the true bactericidal action of the antimicrobial active (denatured alcohol) of the test formulation. Sterile saline was also used as a viability control in place of the test sample formulation for each individual organism.

Appropriate dilutions were plated in duplicate, and 20-25 mL of appropriate agar medium tempered to 45° C. was added. The agar was allowed to solidify and then incubated at 36-38° C. for 24 hours minimum for aerobes and 2-3 days minimum under anaerobic conditions for anaerobes. At the end of each incubation period, the plates were counted for each organism, and the average counts of two plates was determined and expressed in CFU/mL. The percent recovery of the neutralized test material versus the saline controls was calculated. A suitable recovery was one that provided at least 50% of the sterile saline viability control. If there was at least 50% recovery for the neutralized test formulation compared to the saline viability control, then the neutralizer was considered effective.

Results from the neutralizer effectiveness tests are reported in Table 35, below.

TABLE 35
Neutralizer Effectiveness Results

| | | | Sterile | | |
|---|---|---|---|---|---|
| | | Test Sample | Saline | | |
| | | plus | Viability | Neutralizer | |
| | ATCC | Neutralizer | Control | Efficacy | |
| | | Average | Average | at | Valid |
| Test Organism | Number | CFU | CFU | % Recovery | Dilution |
| Haemophilus | 33391 | 34 | 30 | 113.3 | 1:100 |
| influenzae | | | | | |
| Bacteraides | 25285 | 59 | 61 | 96.7 | 1:100 |
| fragilis | | | | | |
| Enterobacter | 13047 | 61 | 70 | 87.1 | 1:10 |
| cloacae | | | | | |
| Burkholderia | 25416 | 80 | 83 | 96.4 | 1:10 |
| cepacia | | | | | |
| Burkholderia | 25608 | 51 | 54 | 94.4 | 1:10 |
| cepacia | | | | | |
| Escherichia coli | 11775 | 55 | 61 | 90.2 | 1:10 |
| Escherichia coli | 25922 | 43 | 44 | 97.7 | 1:10 |
| Klebsiella | 13383 | 87 | 86 | 101.2 | 1:10 |
| pneumoniae | | | | | |
| Klebsiella | 27736 | 56 | 58 | 96.6 | 1:10 |
| pneumoniae | | | | | |
| Pseudomonas | 15442 | 81 | 83 | 98.0 | 1:10 |
| aeruginosa | | | | | |
| Pseudomonas | 27853 | 49 | 49 | 100.0 | 1:10 |
| aeruginosa | | | | | |
| Serrotia | 8100 | 87 | 81 | 107.4 | 1:10 |
| marcescens | | | | | |
| Serrotia | 14756 | 68 | 61 | 111.5 | 1:10 |
| marcescens | | | | | |
| Campylobacter | 33291 | 87 | 90 | 96.7 | 1:100 |
| jejuni | | | | | |
| Campylobacter | 49943 | 88 | 89 | 98.9 | 1:100 |
| jejuni | | | | | |
| Salmonella | 13076 | 82 | 83 | 98.8 | 1:10 |
| enterica | | | | | |
| serovar | | | | | |
| enteritidis | | | | | |
| Salmonella | 14028 | 38 | 40 | 95.0 | 1:10 |
| enterica | | | | | |
| serovar | | | | | |
| typhimurium | | | | | |
| Shigella sonnei | 9290 | 58 | 67 | 86.6 | 1:10 |
| Shigella sonnei | 25931 | 33 | 33 | 100.0 | 1:10 |
| Enterocaccus | 19433 | 85 | 86 | 98.8 | 1:10 |
| faecalis | | | | | |
| Enterocaccus | 29212 | 58 | 62 | 93.5 | 1:10 |
| faecalis | | | | | |
| Staphyiococcus | 6538 | 53 | 49 | 108.2 | |
| aureus | | | | | |
| Staphyiococcus | 29213 | 86 | 85 | 101.2 | 1:10 |
| aureus | | | | | |
| Methicillin- | 33591 | 82 | 84 | 97.6 | 1:10 |
| resistant | | | | | |
| Staphyiococcus | | | | | |
| aureus | | | | | |
| Methicillin- | 33592 | 49 | 53 | 92.5 | 1.10 |
| resistant | | | | | |
| Staphyiococcus | | | | | 1:10 |
| aureus | | | | | |
| Streptocauccus | 14289 | 77 | 82 | 93.9 | 1:100 |
| pyogenes | | | | | |
| Streptocauccus | 19615 | 56 | 55 | 101.8 | 1:100 |
| pyogenes | | | | | |
| Listeria | 7644 | 78 | 81 | 96.3 | 1:10 |
| monocytogenes | | | | | |
| Listeria | 19115 | 64 | 65 | 98.5 | 1:10 |
| monocytogenes | | | | | |
| Streptocauccus | 6303 | 30 | 34 | 88.2 | 1:100 |
| pneumoniae | | | | | |
| Streptocauccus | 49619 | 40 | 45 | 88.9 | 1:100 |
| pneumoniae | | | | | |

To test the toxicity of the neutralizer, 1 mL sterile saline was added to 9 mL D/E neutralizing broth. The appropriate volume of test organism was added to achieve <100 organisms, and the mixture was allowed to stand for approximately 15 minutes. After 15 minutes, a 1 mL aliquot for aerobes and a 0.1 mL aliquot for anaerobes were plated in duplicate using the media appropriate for each test organism. Plates were incubated at 36-38° C. for 24 hours minimum for aerobes and 2-3 days minimum under anaerobic conditions for anaerobes. Neutralizer was considered non-toxic up to and including the concentration at which organism recovery was within +/−50% of sterile saline viability control counts.

Results from the neutralizer toxicity tests are reported in Table 36, below.

TABLE 36
Neutralizer Toxicity Results for D/E Neutralizing Broth

| | | Sterile | | | |
|---|---|---|---|---|---|
| | | Saline | | | |
| | | plus | Sterile | | |
| | | D/E | Saline | | |
| | | Neutralizer | Viability | Neutralizer | |
| | | Broth | Control | Toxicity | |
| | ATCC | Average | Average | % | Valid |
| Test Organisrn | Number | CFU | CFU | Recovery | Dilution |
| Haemophilus | 33391 | 34 | 30 | 113.3 | 1:100 |
| influenzae | | | | | |
| Bacteraides | 25285 | 61 | 61 | 100.0 | 1:100 |
| fragilis | | | | | |
| Enterobacter | 13047 | 71 | 70 | 101.4 | 1:10 |
| cloacae | | | | | |
| Burkholderia | 25416 | 81 | 83 | 97.6 | 1:10 |
| cepacia | | | | | |
| Burkholderia | 25608 | 54 | 54 | 100.0 | 1:10 |
| cepacia | | | | | |
| Escherichia | 11775 | 57 | 61 | 93.4 | 1:10 |
| coli | | | | | |
| Escherichia | 25922 | 42 | 44 | 95.5 | 1:10 |
| coli | | | | | |
| Klebsiella | 13883 | 81 | 86 | 94.2 | 1:10 |
| pneumoniae | | | | | |
| Klebsiella | 27736 | 56 | 58 | 96.6 | 1:10 |
| pneumoniae | | | | | |
| Pseudomonas | 15442 | 80 | 83 | 96.4 | 1:10 |
| aeruginosa | | | | | |
| Pseudomonas | 27853 | 49 | 49 | 100.0 | 1:10 |
| aeruginosa | | | | | |
| Serrotia | 8100 | 89 | 81 | 109.9 | |
| marcescens | | | | | |
| Serrotia | 14756 | 63 | 61 | 103.3 | 1:10 |
| marcescens | | | | | |
| Campylobacter | 33291 | 84 | 90 | 93.3 | 1:100 |
| jejuni | | | | | |
| Campylobacter | 49943 | 89 | 89 | 100.0 | 1:100 |
| jejuni | | | | | |
| Salmonella | 13076 | 82 | 83 | 98.8 | 1:10 |
| enterica | | | | | |
| serovar | | | | | |
| enteritidis | | | | | |
| Salmonella | 14028 | 38 | 40 | 95.0 | 1:10 |
| enterica | | | | | |
| serovar | | | | | |
| typhimurium | | | | | |
| Shigella sonnei | 9290 | 62 | 67 | 92.5 | 1.10 |
| Shigella sonnei | 25931 | 31 | 33 | 93.9 | 1:10 |
| Enterocaccus | 19433 | 81 | 86 | 94.2 | 1:10 |
| faecalis | | | | | |
| Enterocaccus | 29212 | 62 | 62 | 100.0 | 1:10 |
| faecalis | | | | | |
| Staphyiococcus | 6538 | 54 | 49 | 110.2 | 1:10 |
| aureus | | | | | |
| Staphyiococcus | 29213 | 84 | 85 | 98.8 | 1:10 |
| aureus | | | | | |
| Methicillin- | 33591 | 82 | 84 | 97.6 | 1:10 |
| resistant | | | | | |
| Staphyiococcus | | | | | |
| aureus | | | | | |
| Methicillin- | 33592 | 51 | 55 | 96.2 | 1:10 |
| resistant | | | | | |
| Staphyiococcus | | | | | |
| aureus | | | | | |
| Streptocauccus | 14289 | 83 | 82 | 101.2 | 1:100 |
| pyogenes | | | | | |
| Streptocauccus | 19615 | 51 | 55 | 92.7 | 1:100 |
| pyogenes | | | | | |
| Listeria | 7644 | 81 | 81 | 100.0 | 1:10 |
| monocytogenes | | | | | |
| Listeria | 19115 | 63 | 65 | 96.9 | 1:10 |
| monocytogenes | | | | | |
| Streptocauccus | 6303 | 32 | 34 | 94.1 | 1:100 |
| pneumoniae | | | | | |
| Streptocauccus | 49619 | 40 | 45 | 88.9 | 1:100 |
| pneumoniae | | | | | |

### Example 4

### Exemplary Assays

Assays that can be used to determine the efficacy of any one of the ingredients or any combination of ingredients or compositions having said combination of ingredients disclosed throughout the specification and claims can be determined by methods known to those of ordinary skill in the art. The following are non-limiting assays that can be used in the context of the present invention. It should be recognized that other testing procedures can be used, including, for example, objective and subjective procedures.

Antibacterial and Antifungal Susceptibility Testing Assays:

Agar disk-diffusion method. Agar plates are inoculated with a standardized inoculum of a test microorganism. Then, filter paper discs, containing the test compound at a desired concentration, are placed on the agar surface. The Petri dishes are incubated under suitable conditions. Antimicrobial agent diffuses into the agar and inhibits germination and growth of the test microorganism and then the diameters of inhibition growth zones are measured.

Antimicrobial gradient method. Inert and non-porous reagent strips with a predefined gradient of antimicrobial agent, covering a continuous concentration range, are used for the determination of precise minimum inhibitor concentration (MIC) values of an antimicrobial agent against an organism strain. When the strip is applied to the surface of an agar plate inoculated with the test strain, there is an instantaneous release of the antimicrobial gradient from the carrier to the agar to form a stable and continuous gradient beneath and in the immediate vicinity of the strip. The bacterial growth becomes visible after incubation and a symmetrical inhibition ellipse centered along the strip is seen. When an even lawn of growth is distinctly visible, the MIC value is read from the scale in terms of μg/mL where the ellipse edge intersects the strip. Incubation and reading times have been determined based on the intrinsic growth characteristics of the organism and the specific incubation conditions.

Agar well diffusion method. An agar plate surface is inoculated by spreading a volume of the microbial inoculum over the entire agar surface. Then, a hole with a diameter of 6 to 8 mm is punched aseptically with a sterile cork borer or a tip, and a volume (20-100 μL) of the antimicrobial agent or extract solution at desired concentration is introduced into the well. Then, agar plates are incubated under suitable conditions depending upon the test microorganism. The antimicrobial agent diffuses in the agar medium and inhibits the growth of the microbial strain tested, and inhibition growth zones are measured.

Broth dilution method. Dilutions of the antimicrobial agent in a liquid growth medium are dispensed into tubes containing a minimum volume of liquid growth medium. Then, each tube or well is inoculated with a microbial inoculum prepared in the same medium after dilution of standardized microbial suspension. After well-mixing, the inoculated tubes are incubated under suitable conditions depending upon the test microorganism, and microbial growth is measured. The MIC is the lowest concentration of antimicrobial agent that completely inhibits growth of the organism in tubes as detected by the unaided eye.

Agar dilution method. Varying desired concentrations of the antimicrobial agent are incorporated into an agar medium, using serial dilutions, followed by the inoculation of a defined microbial inoculum onto the agar plate surface. The MIC endpoint is recorded as the lowest concentration of antimicrobial agent that completely inhibits growth under suitable incubation conditions.

Time-kill test. Antimicrobial agent is added to culture medium containing a bacterial suspension. Incubation is done under suitable conditions for varied time intervals. Then, the percentage of dead cells is calculated relative to a growth control by determining the number of living cells (CFU/mL) using the agar plate count method. Generally, the bactericidal effect is obtained with a lethality percentage of 90% for 6 h, which is equivalent to 99.9% of lethality for 24 h.

ATP bioluminescence assay. ATP bioluminescence assay is based on the capacity to measure adenosine triphosphate (ATP) produced by bacteria or fungi. As ATP is the chemical form of energy of all living cells, it is present in more or less a constant amount in a cell. Therefore, its quantification is used to estimate the microbial population in a sample. D-luciferin in the presence of the ATP undergoes conversion by luciferase to oxyluciferin that generates light. The quantity of the emitted light is measured by a luminometer and expressed as relative light unit (RLU) which can be converted into RLU/mole of ATP. Thus, there is a linear relationship between cell viability and luminescence measured.

Flow cytofluorometric method. Growth medium is inoculated with a standardized inoculum of a test microorganism. Then, antimicrobial agents is added at a desired concentration. The Petri dishes are incubated under suitable conditions. Flow cytometry is then used to measure membrane integrity and other indicators of microbial viability on a cell-by-cell basis, and a MIC can be calculated.

Virucidal Efficacy Testing Assays:

Suspension time-kill test for viruses. A prepared viral inoculum and a test anti-viral product are combined and serially diluted in appropriate media. Each dilution is plated to host cell monolayers plated in wells. Media is added to each well, and the host cell-virus system is allowed to incubate for the appropriate time. At the close of the incubation time, the assay is scored using standard cell culture methods. Each well in the tray is examined under microscope for the presence of cytopathic effects (CPE) of infection such as cell rounding, sloughing, and monolayer degradation. Cytotoxicity control wells are examined for damage caused by the test product. Confirmatory assays are used as necessary. The Spearman-Karber method, or another appropriate statistical method, is used to quantify the amount of infectious virus present in the assay.

Surface time-kill test for viruses. A prepared viral inoculum is spread over the entire surface of a glass Petri dish carrier and allowed to dry. At the conclusion of the dry time the test anti-viral agent is applied to the viral film to form a suspension. Upon closure of the study contact time, suspensions are harvested by use of a cell scraper and neutralized by a method most suitable for the active ingredient(s) present in the anti-viral substance. The suspensions are serially diluted in appropriate media. Each dilution is plated to host cell monolayers plated in wells. Media is added to each well, and the host cell-virus system is allowed to incubate for the appropriate time. At the close of the incubation time, the assay is scored using standard cell culture methods. Each well in the tray is examined under microscope for the presence of cytopathic effects (CPE) of infection such as cell rounding, sloughing, and monolayer degradation. Cytotoxicity control wells are examined for damage caused by the test product. Confirmatory assays are used as necessary. The Spearman-Karber method, or another appropriate statistical method, is used to quantify the amount of infectious virus present in the assay.

Antioxidant (AO) Assay: An antioxidant assay can be performed on skin cells (e.g., epidermal keratinocytes, fibroblasts, and/or dermal endothelial cells) to determine the ability of any one of the active ingredients, combination of ingredients, or compositions having said combinations disclosed in the specification to provide anti-oxidant capacity (TEAC) by inhibiting the oxidation of ABTS® (2,2′-azino-di-[3-ethylbenzthiazoline sulphonate]) to ABTS®+ by metmyoglobin. The antioxidant system of living organisms can include enzymes such as superoxide dismutase, catalase, and glutathione peroxidase; macromolecules such as albumin, ceruloplasmin, and ferritin; and an array of small molecules, including ascorbic acid, α-tocopherol, β-carotene, reduced glutathione, uric acid, and bilirubin. The sum of endogenous and food-derived antioxidants represents the total antioxidant activity of the extracellular fluid. Cooperation of all the different antioxidants can provide greater protection against attack by reactive oxygen or nitrogen radicals, than any single compound alone. Thus, the overall antioxidant capacity may give more relevant biological information compared to that obtained by the measurement of individual components, as it considers the cumulative effect of all antioxidants present in plasma and body fluids. The capacity of the ingredients in the composition to prevent ABTS oxidation can be compared with that of Trolox, a water-soluble tocopherol analogue, and was quantified as molar Trolox equivalents. Anti-Oxidant capacity kit #709001 from Cayman Chemical (Ann Arbor, Mich. USA) can be used to measure the total anti-oxidant capacity.

B16 Pigmentation Assay: Melanogenesis is the process by which melanocytes produce melanin, a naturally produced pigment that imparts color to skin, hair, and eyes. Inhibiting melanogenesis is beneficial to prevent skin darkening and lighten dark spots associated with aging. B16 pigmentation assays utilize B16-F1 melanocytes (ATCC), an immortalized mouse melanoma cell line, to analyze the effect of active ingredients on melanogenesis. B16-F1 melanocytes are cultivated in standard DMEM growth medium with 10% fetal bovine serum (Mediatech) at 37° C. in 10% CO₂ and then treated with any one of the active ingredients, combination of ingredients, or compositions having said combinations disclosed in the specification for 6 days. The endpoint of these assays are a spectrophotometric measurement of melanin production and cellular viability.

Collagen Stimulation Assay: A collagen stimulation assay can be used to determine the ability of any one of the active ingredients, combination of ingredients, or compositions having said combinations disclosed in the specification to increase expression of procollagen-1, a precursor to collagen. Collagens (types I, II, III, IV and V) can be synthesized as precursor molecules called procollagens. These precursor molecules can contain additional peptide sequences, usually called “propeptides”, at both the amino-terminal and the carboxy-terminal ends. During cellular expression and secretion, procollagens can be assembled in the trimeric form and then cleaved at specific N- and C-terminal sites by specific endopeptidases, generating three fragments: procollagen-1 N-terminal propeptide (PINP), Type I collagen, and procollagen-1 carboxy-terminal propeptide (PICP).

The function of the propeptides is to facilitate the winding of procollagen molecules into a triple-helical conformation within the endoplasmic reticulum. The propeptides can be cleaved off from the collagen triple helix molecule during its secretion, after which the triple helix collagens polymerize into extracellular collagen fibrils. Thus, the amount of the free propeptides reflects stoichiometrically the amount of collagen molecules synthesized (a relationship analogous to that between the carboxy-terminal peptide of proinsulin and the endogenously produced insulin). Collagen is an extracellular matrix protein critical for skin structure. Increased synthesis of collagen helps improve skin firmness and elasticity.

Quantitative detection of PICP in fibroblast cell extracts and culture supernatants can be performed with an enzyme immunoassay kit (e.g., Takara #MK101) to assess the effects of the ingredients on the synthesis of PICP in skin. This bioassay can be used to examine effects on the production of procollagen peptide (a precursor to collagen) by human epidermal fibroblasts. The endpoint of this assay can be a spectrophotometric measurement that reflects the presence of procollagen peptide and cellular viability. The assay employs the quantitative sandwich enzyme immunoassay technique whereby a monoclonal antibody specific for procollagen peptide was pre-coated onto a microplate. Standards and samples can be pipetted into the wells and any procollagen peptide present was bound by the immobilized antibody. After washing away any unbound substances, an enzyme-linked polyclonal antibody specific for procollagen peptide can be added to the wells. Following a wash to remove any unbound antibody-enzyme reagent, a substrate solution can be added to the wells and color was developed in proportion to the amount of procollagen peptide bound in the initial step. Color development was stopped and the intensity of the color at 450 nm was measured using a microplate reader.

For generation of samples and controls, subconfluent normal human adult epidermal fibroblasts (Cascade Biologics) can be cultivated in standard DMEM growth medium with 10% fetal bovine serum (Mediatech) at 37° C. in 10% CO2. The cells can be treated with each of the tested ingredients and controls for 3 days. Following incubation, cell culture medium can be collected and the amount of Type I procollagen peptide secretion was quantified using the sandwich enzyme linked immuno-sorbant assay (ELISA) from Takara (#MK101) as explained above.

Elastin Stimulation Assay: Elastin is a connective tissue protein that helps skin resume shape after stretching or contracting. Elastin is also an important load-bearing protein used in places where mechanical energy is required to be stored. Elastin is made by linking many soluble tropoelastin protein molecules, in a reaction catalyzed by lysyl oxidase. Elastin secretion and elastin fibers can be monitored in cultured human fibroblasts by staining of cultured human fibroblasts using immunofluorescent antibodies directed against elastin by a direct ELISA sandwich method. A Meso Scale Discovery system SECTOR 2400 Imaging system can be used to analyze the results. Changes in elastin secretion and elastin fibers caused by one or more ingredients in the composition can be determined by incubating cultured human fibroblasts with the active ingredient for a period of time before probing the cells or a lysate thereof with antibodies directed against elastin.

Laminin Stimulation Assay: Laminin is a major protein in the dermal-epidermal junction (DEJ) (also referred to as the basement membrane). The DEJ is located between the dermis and the epidermis interlocks forming fingerlike projections called rete ridges. The cells of the epidermis receive their nutrients from the blood vessels in the dermis. The rete ridges increase the surface area of the epidermis that is exposed to these blood vessels and the needed nutrients. The DEJ provides adhesion of the two tissue compartments and governs the structural integrity of the skin. Laminin is a structural glycoprotein located in the DEJ. Together with fibronectin, laminin is considered the glue that holds the cells together, and both are secreted by dermal fibroblasts to help facilitate intra- and inter-cellular adhesion of the epidermal calls to the DEJ.

Laminin secretion can be monitored by quantifying laminin in cell supernatants of cultured human fibroblasts treated for 3 days with culture medium with or without 1.0% final concentration of the test ingredient(s). Following incubation, laminin content can be measured using immunofluorescent antibodies directed against each protein in an enzyme linked immuno-sorbant assay (ELISA).

Matrix Metalloproteinase 1 Enzyme Activity (MMP-1) Assay: MMPs are extracellular proteases that play a role in many normal and disease states by virtue of their broad substrate specificity. MMP-1 substrates include collagen IV. The Molecular Probes Enz/Chek Gelatinase/Collagenase Assay kit (#E12055), can be used to detect MMP-1 protease activity, and utilizes a fluorogenic gelatin substrate and tests proteolytic cleavage of the substrate by purified MMP-1 enzyme. Upon proteolytic cleavage of the substrate, bright green fluorescence is revealed and can be monitored using a fluorescent microplate reader to measure enzymatic activity. Test materials can be incubated in the presence or absence of the purified enzyme and substrate to determine their protease inhibitor capacity.

Matrix Metalloproteinase 3 and 9 Enzyme Activity (MMP-3; MMP-9) Assay: MMPs are extracellular proteases that play a role in many normal and disease states by virtue of their broad substrate specificity. MMP-3 substrates include collagens, fibronectins, and laminin; while MMP-9 substrates include collagen VII, fibronectins and laminin. Colorimetric Drug Discovery kits from BioMol International for MMP-3 (AK-400) and MMP-9 (AK-410) can be used to measure protease activity of MMPs using a thiopeptide as a chromogenic substrate (Ac-PLG-[2-mercapto-4-methyl-pentanoyl]-LG-OC2H5)5,6. The MMP cleavage site peptide bond is replaced by a thioester bond in the thiopeptide. Hydrolysis of this bond by an MMP produces a sulfhydryl group, which reacts with DTNB [5,5′-dithiobis(2-nitrobenzoic acid), Ellman's reagent] to form 2-nitro-5-thiobenzoic acid, which can be detected by its absorbance at 412 nm (ε=13,600 M-1 cm-1 at pH 6.0 and above 7).

Lipoxygenase (LO) Assay: A lipoxygenase assay can be used to determine the ability of any one of the active ingredients, combination of ingredients, or compositions having said combinations disclosed in the specification to inhibit lipoxygenase (LO) expression. LOs are non-heme iron-containing dioxygenases that catalyze the addition of molecular oxygen to fatty acids. Linoleate and arachidonate are the main substrates for LOs in plants and animals. Arachadonic acid may then be converted to hydroxyeicosotrienenoic (HETE) acid derivatives, that are subsequently converted to leukotrienes, potent inflammatory mediators. An accurate and convenient method for screening lipoxygenase inhibitors can be performed by measuring the hydroperoxides generated from the incubation of a lipoxygenase (5-, 12-, or 15-LO) with arachidonic acid. The Colorimetric LO Inhibitor screening kit (#760700, Cayman Chemical) can be used to determine the ability of ingredients of the composition to inhibit enzyme activity.

Purified 15-lipoxygenase and test ingredients can be mixed in assay buffer and incubated with shaking for 10 min at room temperature. Following incubation, arachidonic acid can be added to initiate the reaction and the mixtures were incubated for an additional 10 min at room temperature. Colorimetric substrate can be added to terminate catalysis and color progression was evaluated by fluorescence plate reading at 490 nm. The percent inhibition of lipoxyganse activity can be calculated compared to non-treated controls to determine the ability of ingredients of the composition to inhibit the activity of purified enzyme.

Tumor Necrosis Factor Alpha (TNF-α) Assay: The prototype ligand of the TNF superfamily, TNF-α, is a pleiotropic cytokine that plays a central role in inflammation. Increase in its expression is associated with an up regulation in pro-inflammatory activity. The bioassay can be used to analyze the effect of ingredients of the composition on the production of TNF-α by human epidermal keratinocytes. The endpoint of this assay can be a spectrophotometric measurement that reflects the presence of TNF-α and cellular viability. The assay can employ the quantitative sandwich enzyme immunoassay technique whereby a monoclonal antibody specific for TNF-α had been pre-coated onto a microplate.

Standards and samples can be pipetted into wells of the microplate and any TNF-α present was bound by the immobilized antibody. After washing away any unbound substances, an enzyme-linked polyclonal antibody specific for TNF-α can be added to the wells. Following a wash to remove any unbound antibody-enzyme reagent, a substrate solution can be added to the wells and color developed in proportion to the amount of TNF-α bound in the initial step using a microplate reader for detection at 450 nm. The color development can be stopped and the intensity of the color can be measured. Subconfluent normal human adult keratinocytes (Cascade Biologics) cultivated in EPILIFE™ standard growth medium (Cascade Biologics) at 37° C. in 5% CO₂ can be treated with phorbol 12-myristate 13-acetate (PMA, 10 ng/ml, Sigma Chemical, #P1585-1MG) and of ingredients of the composition or no test ingredient (for negative control) for 6 hours. PMA can be shown to cause a dramatic increase in TNF-α secretion which peaks at 6 hours after treatment. Following incubation, cell culture medium can be collected and the amount of TNF-α secretion quantified using a sandwich enzyme linked immuno-sorbant assay (ELISA) from R&D Systems (#DTA00C).

Elastase Assay: ENZCHEK® Elastase Assay (Kit #E-12056) from Molecular Probes (Eugene, Oreg. USA) can be used as an in vitro enzyme inhibition assay for measuring inhibition of elastase activity in the presence of ingredients of the composition. The EnzChek kit can contain soluble bovine neck ligament elastin that is labeled with dye such that the conjugate's fluorescence is quenched. The non-fluorescent substrate can be digested by elastase or other proteases to yield highly fluorescent fragments. The resulting increase in fluorescence can be monitored with a fluorescence microplate reader. Digestion products from the elastin substrate can have absorption maxima at ˜505 nm and fluorescence emission maxima at ˜515 nm. The peptide, N-methoxysuccinyl-Ala-Ala-Pro-Val-chloromethyl ketone, can be used as a selective, collective inhibitor of elastase for a positive control when utilizing the EnzChek Elastase Assay Kit for screening for elastase inhibitors.

Fibronectin Stimulation Assay: Fibronectin is a major protein in the dermal-epidermal junction (DEJ) (also referred to as the basement membrane). The DEJ is located between the dermis and the epidermis interlocks forming fingerlike projections called rete ridges. The cells of the epidermis receive their nutrients from the blood vessels in the dermis. The rete ridges increase the surface area of the epidermis that is exposed to these blood vessels and the needed nutrients. The DEJ provides adhesion of the two tissue compartments and governs the structural integrity of the skin. Fibronectin is a structural glycoprotein located in the DEJ. Together with laminin, fibronectin is considered the glue that holds the cells together, and both are secreted by dermal fibroblasts to help facilitate intra- and inter-cellular adhesion of the epidermal calls to the DEJ.

Fibronectin secretion can be monitored by quantifying fibronectin in cell supernatants of cultured human fibroblasts treated for 3 days with culture medium with or without 1.0% final concentration of the test ingredient(s). Following incubation, fibronectin content can be measured using immunofluorescent antibodies directed against each protein in an enzyme linked immuno-sorbant assay (ELISA).

Lysyl Oxidase Assay: A lysyl oxidase assay can be performed on skin cells (e.g., epidermal keratinocytes, fibroblasts, and/or dermal endothelial cells) to determine the ability of any one of the active ingredients, combination of ingredients, or compositions having said combinations disclosed in the specification to stimulate expression of lysyl oxidase in skin. Lysyl oxidase can catalyze crosslinking of elastin and collagens, thereby providing for a more structurally rigid matrix for skin. By increasing expression of lysyl oxidase, increased cross-linking of elastin and collagens can occur, which can be beneficial in reducing the appearance of fine lines, wrinkles, sagging skin, and/or non-elastic skin.

ORAC Assay: Oxygen Radical Absorption (or Absorbance) Capacity (ORAC) of any one of the active ingredients, combination of ingredients, or compositions having said combinations disclosed in the specification can also be assayed by measuring the antioxidant activity of such ingredients or compositions. Antioxidant activity indicates a capability to reduce oxidizing agents (oxidants). This assay quantifies the degree and length of time it takes to inhibit the action of an oxidizing agent, such as oxygen radicals, that are known to cause damage to cells (e.g., skin cells). The ORAC value of any one of the active ingredients, combination of ingredients, or compositions having said combinations disclosed in the specification can be determined by methods known to those of ordinary skill in the art (see U.S. Publication Nos. 2004/0109905 and 2005/0163880; and commercially available kits such as Zen-Bio ORAC Anti-oxidant Assay kit (#AOX-2)). The Zen-Bio ORAC Anti-oxidant Assay kit measures the loss of fluorescein fluorescence over time due to the peroxyl-radical formation by the breakdown of AAPH (2,2′-axobis-2-methyl propanimidamide, dihydrochloride). Trolox, a water soluble vitamin E analog, serves as positive control inhibition fluorescein decay in a dose dependent manner.

Production of Hyaluronic Acid: Changes in the production of hyaluronic acid (HA) in human dermal fibroblasts due to each of the active ingredients, any one of the combination of ingredients, or compositions having said combinations disclosed in the specification can be measured. HA is a polysaccharide involved in stabilization of the structure of the matrix and is involved in providing turgor pressure to tissue and cells. As one non-limiting example, HA production in treated and non-treated adult human dermal fibroblasts (HDFa) cells can be determined using the Hyaluronan DuoSet ELISA kit from R&D Systems (DY3614). In this assay, for production of samples, subconfluent HDFa cells from Cascade Biologics (C-13-5C) are incubated at 37° C. and 10% CO₂ in starvation medium (0.15% fetal bovine serum and 1% Penicillin Streptomycin solution in Dulbecco's Modified Eagle Medium) for 72 hours prior to treatment. The cells are then incubated with fresh starvation medium with either test compound, positive control (phorbol 12-myristate 13-acetate from Sigma-Aldrich (P1585) and platelet derived growth factor from Sigma-Aldrich (P3201)), or no additive for 24 hours. Media is then collected and frozen at −80° C. until use in the ELISA assay.

Briefly, the ELISA assay employs a quantitative sandwich enzyme immunoassay technique whereby a capture antibody specific for HA can be pre-coated onto a microplate. Standards and media from treated and untreated cells are pipetted into the microplate wells to enable any HA present to be bound by the immobilized antibody. After washing away any unbound substances, an enzyme-linked detection antibody specific for HA is added to the wells. Following a wash to remove any unbound antibody-enzyme reagent, a substrate solution is added to the wells to allow color development in proportion to the amount of HA bound in the initial step. The color development is stopped at a specific time and the intensity of the color at 450 nm can be measured using a microplate reader.

Production of Occludin: Changes in the production of occludin in keratinocytes due to each of the active ingredients, any one of the combination of ingredients, or compositions having said combinations disclosed in the specification can be measured. Occludin is a protein critical to the formulation of tight junctions and the skin's moisture barrier function. A non-limiting example of how occludin production in treated and non-treated keratinocytes can be determined is by the use of a bioassay that analyzes occludin concentration in keratinocyte cell lysates. The bioassay can be performed using PROTEINSIMPLE® SIMON™ western blotting protocol. For the samples, adult human epidermal keratinocytes (HEKa) from Life Technologies (C-005-5C) can be grown at 37° C. and 5% CO₂ for 24 hours in EPILIFE™ growth media with calcium from Life Technologies (M-EP-500-CA) supplemented with Keratinocyte Growth Supplement (HKGS) from Life Technologies (S-101-5). HEKa are then incubated in growth medium with test compound/extract, no compound/extract for negative control, or with 1 mM CaCl₂ for positive control for 24 to 48 hours. The HEKa are then washed, collected, and stored on ice or colder until lysed on ice using a lysis buffer and sonication. The protein concentrations of the samples can be determined and used to normalize the samples. The lysates are stored at −80° C. until use in the bioassay.

The PROTEINSIMPLE® SIMON™ western blotting bioassay assay employs a quantitative western blotting immunoassay technique using an antibody specific for occludin to quantitatively detect occludin in the test samples. Cell samples are lysed and normalized for protein concentration. Normalized samples and molecular weight standards are then loaded and ran on a denatured protein separation gel using capillary electrophoresis. The proteins in the gel are then immobilized and immunoprobed using a primary antibody specific for occludin. The immobilized proteins are immunoprobed with an enzyme-linked detection antibody that binds the primary antibody. A chemiluminescent substrate solution is then added to the immobilized proteins to allow chemiluminescent development in proportion to the amount of occludin bound in the immobilization. The chemiluminescent development can be stopped at a specific time and the intensity of the chemiluminescent signal can be measured and compared to positive and negative controls.

Keratinocyte Monolayer Permeability: Changes in the permeability of a keratinocyte monolayer due to each of the active ingredients, any one of the combination of ingredients, or compositions having said combinations disclosed in the specification can be measured. Keratinocyte monolayer permeability is a measure of skin barrier integrity. Keratinocyte monolayer permeability in treated and non-treated keratinocytes can be determined using, as a non-limiting example, the In Vitro Vascular Permeability assay by Millipore (ECM642). This assay analyzes endothelial cell adsorption, transport, and permeability. Briefly, adult human epidermal keratinocytes from Life Technologies (C-005-5C) can be seeded onto a porous collagen-coated membrane within a collection well. The keratinocytes are then incubated for 24 hours at 37° C. and 5% CO₂ in Epilife growth media with calcium from Life Technologies (M-EP-500-CA) supplemented with Keratinocyte Growth Supplement (HKGS) from Life Technologies (S-101-5). This incubation time allows the cells to form a monolayer and occlude the membrane pores. The media is then replaced with fresh media with (test sample) or without (non-treated control) test compounds/extracts and the keratinocytes are incubated for an additional 48 hours at 37° C. and 5% CO2. To determine permeability of the keratinocyte monolayer after incubation with/without the test compound/extract, the media is replaced with fresh media containing a high molecular weight Fluorescein isothiocyanate (FITC)-Dextran and the keratinocytes are incubated for 4 hours at 37° C. and 5% CO2. During the 4 hours incubation, FITC can pass through the keratinocytes monolayer and porous membrane into the collection well at a rate proportional to the monolayer's permeability. After the 4 hour incubation, cell viability and the content of FITC in the collection wells can be determined. For the FITC content, the media in the collection well is collected and fluorescence of the media determined at 480 nm (Em) when excited at 520 nm. Percent permeability and percent change in comparison to the non-treated controls can be determined by the following equations: Percent Permeability=((Mean Ex/Em of test sample)/Mean Ex/Em untreated control)*100; Percent Change=Percent Permeability of test sample−Percent Permeability of untreated control.

Mushroom tyrosinase activity assay: In mammalian cells, tyrosinase catalyzes two steps in the multi-step biosynthesis of melanin pigments from tyrosine (and from the polymerization of dopachrome). Tyrosinase is localized in melanocytes and produces melanin (aromatic quinone compounds) that imparts color to skin, hair, and eyes. Purified mushroom tyrosinase (Sigma) can be incubated with its substrate L-Dopa (Fisher) in the presence or absence of each of the active ingredients, any one of the combination of ingredients, or compositions having said combinations disclosed in the specification. Pigment formation can be evaluated by colorimetric plate reading at 490 nm. The percent inhibition of mushroom tyrosinase activity can be calculated compared to non-treated controls to determine the ability of test ingredients or combinations thereof to inhibit the activity of purified enzyme. Test extract inhibition was compared with that of kojic acid (Sigma).

Cyclooxygenase (COX) Assay: An in vitro cyclooxygenase-1 and -2 (COX-1, -2) inhibition assay. COX is a bifunctional enzyme exhibiting both cyclooxygenase and peroxidase activities. The cyclooxygenase activity converts arachidonic acid to a hydroperoxy endoperoxide (Prostaglandin G2; PGG2) and the peroxidase component reduces the endoperoxide (Prostaglandin H2; PGH2) to the corresponding alcohol, the precursor of prostaglandins, thromboxanes, and prostacyclins. This COX Inhibitor screening assay measures the peroxidase component of cyclooxygenases. The peroxidase activity is assayed colorimetrically by monitoring the appearance of oxidized N,N,N′,N′-tetramethyl-p-phenylenediamine (TMPD). This inhibitor screening assay includes both COX-1 and COX-2 enzymes in order to screen isozyme-specific inhibitors. The Colormetric COX (ovine) Inhibitor screening assay (#760111, Cayman Chemical) can be used to analyze the effects of each of the active ingredients, any one of the combination of ingredients, or compositions having said combinations disclosed in the specification on the activity of purified cyclooxygnase enzyme (COX-1 or COX-2). According to manufacturer instructions, purified enzyme, heme and test extracts can be mixed in assay buffer and incubated with shaking for 15 min at room temperature. Following incubation, arachidonic acid and colorimetric substrate can be added to initiate the reaction. Color progression can be evaluated by colorimetric plate reading at 590 nm. The percent inhibition of COX-1 or COX-2 activity can be calculated compared to non-treated controls to determine the ability of test extracts to inhibit the activity of purified enzyme.

Oil Control Assay: An assay to measure reduction of sebum secretion from sebaceous glands and/or reduction of sebum production from sebaceous glands can be assayed by using standard techniques known to those having ordinary skill in the art. In one instance, the forehead can be used. Each of the active ingredients, any one of the combination of ingredients, or compositions having said combinations disclosed in the specification can be applied to one portion of the forehead once or twice daily for a set period of days (e.g., 1, 2, 3, 4, 5, 6, 7, 8, 9, 10, 11, 12, 13, 14, or more days), while another portion of the forehead is not treated with the composition. After the set period of days expires, then sebum secretion can be assayed by application of fine blotting paper to the treated and untreated forehead skin. This is done by first removing any sebum from the treated and untreated areas with moist and dry cloths. Blotting paper can then be applied to the treated and untreated areas of the forehead, and an elastic band can be placed around the forehead to gently press the blotting paper onto the skin. After 2 hours the blotting papers can be removed, allowed to dry and then transilluminated. Darker blotting paper correlates with more sebum secretion (or lighter blotting paper correlates with reduced sebum secretion.

Erythema Assay: An assay to measure the reduction of skin redness can be evaluated using a Minolta Chromometer. Skin erythema may be induced by applying a 0.2% solution of sodium dodecyl sulfate on the forearm of a subject. The area is protected by an occlusive patch for 24 hrs. After 24 hrs, the patch is removed and the irritation-induced redness can be assessed using the a* values of the Minolta Chroma Meter. The a* value measures changes in skin color in the red region. Immediately after reading, the area is treated with the active ingredients, any one of the combination of ingredients, or compositions having said combinations disclosed in the specification. Repeat measurements can be taken at regular intervals to determine the formula's ability to reduce redness and irritation.

Skin Moisture/Hydration Assay: Skin moisture/hydration benefits can be measured by using impedance measurements with the Nova Dermal Phase Meter. The impedance meter measures changes in skin moisture content. The outer layer of the skin has distinct electrical properties. When skin is dry it conducts electricity very poorly. As it becomes more hydrated increasing conductivity results. Consequently, changes in skin impedance (related to conductivity) can be used to assess changes in skin hydration. The unit can be calibrated according to instrument instructions for each testing day. A notation of temperature and relative humidity can also be made. Subjects can be evaluated as follows: prior to measurement they can equilibrate in a room with defined humidity (e.g., 30-50%) and temperature (e.g., 68-72° C.). Three separate impedance readings can be taken on each side of the face, recorded, and averaged. The T5 setting can be used on the impedance meter which averages the impedance values of every five seconds application to the face. Changes can be reported with statistical variance and significance. Each of the active ingredients, any one of the combination of ingredients, or compositions having said combinations disclosed in the specification can be assayed according to this process.

Skin Clarity and Reduction in Freckles and Age Spots Assay: Skin clarity and the reduction in freckles and age spots can be evaluated using a Minolta Chromometer. Changes in skin color can be assessed to determine irritation potential due to product treatment using the a* values of the Minolta Chroma Meter. The a* value measures changes in skin color in the red region. This is used to determine whether each of the active ingredients, any one of the combination of ingredients, or compositions having said combinations disclosed in the specification is inducing irritation. The measurements can be made on each side of the face and averaged, as left and right facial values. Skin clarity can also be measured using the Minolta Meter. The measurement is a combination of the a*, b, and L values of the Minolta Meter and is related to skin brightness, and correlates well with skin smoothness and hydration. Skin reading is taken as above. In one non-limiting aspect, skin clarity can be described as L/C where C is chroma and is defined as (a2+b2)1/2.

Skin Dryness, Surface Fine Lines, Skin Smoothness, and Skin Tone Assay: Skin dryness, surface fine lines, skin smoothness, and skin tone can be evaluated with clinical grading techniques. For example, clinical grading of skin dryness can be determined by a five point standard Kligman Scale: (0) skin is soft and moist; (1) skin appears normal with no visible dryness; (2) skin feels slightly dry to the touch with no visible flaking; (3) skin feels dry, tough, and has a whitish appearance with some scaling; and (4) skin feels very dry, rough, and has a whitish appearance with scaling. Evaluations can be made independently by two clinicians and averaged.

Clinical Grading of Skin Tone Assay: Clinical grading of skin tone can be performed via a ten point analog numerical scale: (10) even skin of uniform, pinkish brown color. No dark, erythremic, or scaly patches upon examination with a hand held magnifying lens. Microtexture of the skin very uniform upon touch; (7) even skin tone observed without magnification. No scaly areas, but slight discolorations either due to pigmentation or erythema. No discolorations more than 1 cm in diameter; (4) both skin discoloration and uneven texture easily noticeable. Slight scaliness. Skin rough to the touch in some areas; and (1) uneven skin coloration and texture. Numerous areas of scaliness and discoloration, either hypopigmented, erythremic or dark spots. Large areas of uneven color more than 1 cm in diameter. Evaluations were made independently by two clinicians and averaged.

Clinical Grading of Skin Smoothness Assay: Clinical grading of skin smoothness can be analyzed via a ten point analog numerical scale: (10) smooth, skin is moist and glistening, no resistance upon dragging finger across surface; (7) somewhat smooth, slight resistance; (4) rough, visibly altered, friction upon rubbing; and (1) rough, flaky, uneven surface. Evaluations were made independently by two clinicians and averaged.

Skin Smoothness and Wrinkle Reduction Assay With Methods Disclosed in Packman et al. (1978): Skin smoothness and wrinkle reduction can also be assessed visually by using the methods disclosed in Packman et al. (1978). For example, at each subject visit, the depth, shallowness and the total number of superficial facial lines (SFLs) of each subject can be carefully scored and recorded. A numerical score was obtained by multiplying a number factor times a depth/width/length factor. Scores are obtained for the eye area and mouth area (left and right sides) and added together as the total wrinkle score.

Appearance of Lines and Wrinkles Assay with Replicas: The appearance of lines and wrinkles on the skin can be evaluated using replicas, which is the impression of the skin's surface. Silicone rubber like material can be used. The replica can be analyzed by image analysis. Changes in the visibility of lines and wrinkles can be objectively quantified via the taking of silicon replicas form the subjects' face and analyzing the replicas image using a computer image analysis system. Replicas can be taken from the eye area and the neck area, and photographed with a digital camera using a low angle incidence lighting. The digital images can be analyzed with an image processing program and are of the replicas covered by wrinkles or fine lines was determined.

Skin Firmness Assay with a Hargens Ballistometer: Skin firmness can be measured using a Hargens ballistometer, a device that evaluates the elasticity and firmness of the skin by dropping a small body onto the skin and recording its first two rebound peaks. The ballistometry is a small lightweight probe with a relatively blunt tip (4 square mm-contact area) was used. The probe penetrates slightly into the skin and results in measurements that are dependent upon the properties of the outer layers of the skin, including the stratum corneum and outer epidermis and some of the dermal layers.

Skin Softness/Suppleness Assay with a Gas Bearing Electrodynamometer: Skin softness/suppleness can be evaluated using the Gas Bearing Electrodynamometer, an instrument that measures the stress/strain properties of the skin. The viscoelastic properties of skin correlate with skin moisturization. Measurements can be obtained on the predetermined site on the cheek area by attaching the probe to the skin surface with double-stick tape. A force of approximately 3.5 gm can be applied parallel to the skin surface and the skin displacement is accurately measured. Skin suppleness can then be calculated and is expressed as DSR (Dynamic Spring Rate in gm/mm).

Surface Contour of the Skin Assay with a Profilometer/Stylus Method: The surface contour of the skin can be measured by using the profilometer/Stylus method. This includes either shining a light or dragging a stylus across the replica surface. The vertical displacement of the stylus can be fed into a computer via a distance transducer, and after scanning a fixed length of replica a cross-sectional analysis of skin profile can be generated as a two-dimensional curve. This scan can be repeated any number of times along a fix axis to generate a simulated 3-D picture of the skin. Ten random sections of the replicas using the stylus technique can be obtained and combined to generate average values. The values of interest include Ra which is the arithmetic mean of all roughness (height) values computed by integrating the profile height relative to the mean profile height. Rt which is the maximum vertical distance between the highest peak and lowest trough, and Rz which is the mean peak amplitude minus the mean peak height. Values are given as a calibrated value in mm. Equipment should be standardized prior to each use by scanning metal standards of know values. Ra Value can be computed by the following equation: Ra=Standardize roughness; lm=the traverse (scan) length; and y=the absolute value of the location of the profile relative to the mean profile height (x-axis).

MELANODERM™ Assay: In other non-limiting aspects, the efficacy of each of the active ingredients, any one of the combination of ingredients, or compositions having said combinations disclosed in the specification can be evaluated by using a skin analog, such as, for example, MELANODERM™. Melanocytes, one of the cells in the skin analog, stain positively when exposed to L-dihydroxyphenyl alanine (L-DOPA), a precursor of melanin. The skin analog, MELANODERM™, can be treated with a variety of bases containing each of the active ingredients, any one of the combination of ingredients, or compositions having said combinations disclosed in the specification or with the base alone as a control. Alternatively, an untreated sample of the skin analog can be used as a control.

Production of Filaggrin: Changes in the production of filaggrin in keratinocytes due to each of the active ingredients, any one of the combination of ingredients, or compositions having said combinations disclosed in the specification can be measured. Filaggrin is the precursor to Natural Moisturizing Factor (NMF) in the skin. Increased NMF increases the moisture content of the skin. Filaggrin production in treated and non-treated keratinocytes can be determined using a bioassay that analyzes filaggrin concentration in keratinocyte cell lysates. A non-limiting example of a bioassay that can be used to quantify filaggrin production is the PROTEINSIMPLE® SIMON™ western blotting protocol. For each sample, normal human epidermal keratinocytes (NHEK) are grown in EPI-200—Mattek EPILIFE™ growth media with calcium from Life Technologies (M-EP-500-CA). NHEK are incubated in growth medium overnight at 37° C. in 5% CO₂ prior to treatment. NHEK are then incubated in growth medium with 1% test compound/extract or no compound/extract (negative control) for 24 to 36 hours. The NHEK can then be washed, collected, and stored on ice or colder until lysed on ice using a lysis buffer and sonication. The protein concentrations of the samples can be determined and used to normalize the samples. The lysates can be stored at −80° C. until use in the quantification assay.

The PROTEINSIMPLE® SIMON™ western blotting bioassay assay employs a quantitative western blotting immunoassay technique using an antibody specific for filaggrin to quantitatively detect filaggrin in the test samples. Cell samples are lysed and normalized for protein concentration. Normalized samples and molecular weight standards can then be loaded and ran on a denatured protein separation gel using capillary electrophoresis. The proteins in the gel are immobilized and immunoprobed using a primary antibody specific for filaggrin. The immobilized proteins can then be immunoprobed with an enzyme-linked detection antibody that binds the primary antibody. A chemiluminescent substrate solution can then be added to the immobilized proteins to allow chemiluminescent development in proportion to the amount of filaggrin bound in the immobilization. The chemiluminescent development is stopped at a specific time and the intensity of the chemiluminescent signal can be measured and compared to positive and negative controls.

Inhibition of Hyaluronidase Activity: Changes in the activity of hyaluronidase due to each of the active ingredients, any one of the combination of ingredients, or compositions having said combinations disclosed in the specification can be measured. Hyaluronidase is an enzyme that degrades HA. HA is a polysaccharide involved in stabilization of the structure of the matrix and is involved in providing turgor pressure to tissue and cells. As one non-limiting example, hyaluronidase activity can be determined using an in vitro protocol modified from Sigma-Aldrich protocol #EC 3.2.1.35. Briefly, hyaluronidase type 1-S from Sigma-Aldrich (H3506) is added to microplate reaction wells containing test compound or controls. Tannic acid can be used as a positive control inhibitor, no test compound can be added for the control enzyme, and wells with test compound or positive control but without hyaluronidase can be used as a background negative control. The wells are incubated at 37° C. for 10 minutes before addition of substrate (HA). Substrate is added and the reactions incubated at 37° C. for 45 minutes. A portion of each reaction solution is then transferred to and gently mixed in a solution of sodium acetate and acetic acid pH 3.75 to stop that portion of the reaction (stopped wells). The stopped wells and the reaction wells should both contain the same volume of solution after addition of the portion of the reaction solution to the stopped wells. Both the reaction wells and the stopped wells are incubated for 10 minutes at room temperature. Absorbance at 600 nm is then measured for both the reaction wells and the stopped wells. Inhibition can be calculated using the following formulas: Inhibitor (or control) activity=(Inhibitor stopped wells absorbance at 600 nm−inhibitor reaction wells absorbance at 600 nm); Initial activity=control enzyme absorbance at 600 nm; Percent Inhibition=[(Initial activity/Inhibitor Activity)*100]−100.

Peroxisome Proliferator-Activated Receptor Gamma (PPAR-γ) Activity: Changes in the activity of PPAR-γ due to each of the active ingredients, any one of the combination of ingredients, or compositions having said combinations disclosed in the specification can be measured. PPAR-γ is a receptor critical for the production of sebum. As one non-limiting example, the activity of PPAR-γ can be determined using a bioassay that analyzes the ability of a test compound or composition to inhibit binding of a ligand. Briefly, fluorescent small-molecule pan-PPAR ligand, FLUORMONE™ Pan-PPAR Green, available from Life Technologies (PV4894), can be used to determine if test compounds or compositions are able to inhibit binding of the ligand to PPAR-γ. The samples wells include PPAR-γ and fluorescent ligand and either: test compound or composition (test); a reference inhibitor, rosiglitazone (positive control); or no test compound (negative control). The wells are incubated for a set period of time to allow the ligand opportunity to bind the PPAR-γ. The fluorescence polarization of each sample well can then be measured and compared to the negative control well to determine the percentage of inhibition by the test compound or composition.

Cytokine Array: Human epidermal keratinocytes are cultured to 70-80% confluency. The media in the plate is aspirated and 0.025% trypsin/EDTA is added. When the cells became rounded, the culture dish is gently tapped to release the cells. The trypsin/EDTA containing cells are removed from the culture dish and neutralized. Cells are centrifuged for 5 min. at 180×g to form a pellet of cells. The supernatant is aspirated. The resulting pellet is resuspended in EPILIFE™ media (Cascade Biologics). The cells are seeded in 6-well plates at approximately 10-20% confluency. After the cells became approximately 80% confluent, the media is aspirated and 1.0 ml of EPILIFE™, along with phorbol 13-Myristate 12-acetate (“PMA”) (a known inducer of inflammation) and the test composition dilutions are added to two replicate wells (i.e., 1.0% (100 μl of 100× stock) and 0.1% (10 μl of 100× stock) test compositions are diluted into a final volume of 1 ml EpiLife Growth Medium). The media is gently swirled to ensure adequate mixing. In addition, 1.0 ml of EPILIFE™ is added to the control wells, with and without additional PMA. The plates are then incubated at 37±1° C. and 5.0±1% CO₂ for approximately 5 hours after dosing. Following this 5-hour incubation, all media is collected in conical tubes and frozen at −70° C.

For analysis, a 16-pad hybridization chamber is attached to 16-pad FAST slides arrayed in triplicate with 16 anti-cytokine antibodies plus experimental controls (Whatman BioSciences), and the slides are placed into a FASTFrame (4 slides per frame) for processing. Arrays are blocked for 15 min. at room temp. using 70 ml S&S Protein Array Blocking buffer (Whatman Schleicher and Scheull). Blocking buffer is removed and 70 ml of each supernatant sample is added to each array. Arrays are incubated for 3 hours at room temp. with gentle agitation. Arrays are washed 3 times with TBS-T. Arrays are treated with 70 ml of an antibody cocktail, containing one biotinylated antibody corresponding to each of the arrayed capture antibodies. Arrays are incubated for 1 hour at room temp. with gentle agitation. Arrays are washed 3 times with TBS-T. Arrays are incubated with 70 ml of a solution containing streptavidin-Cy5 conjugate for 1 hour at room temp. with gentle agitation. Arrays are washed 3 times with TBS-T, quickly rinsed in de-ionized water, and dried.

Slides can be imaged in a Perkin-Elmer ScanArray 4000 confocal fluorescent imaging system. Array images can be saved and analyzed using Imaging Research ArrayVision software. Briefly, spot intensities are determined by subtracting background signal. Spot replicates from each sample condition can be averaged and then compared to the appropriate controls.

Endothelial Tube Formation: Endothelial tube formation is involved in angiogenesis and micro-vessel capillary formation. Capillary formation and angiogenesis may contribute to redness and rosacea of the skin. The ability for endothelial cells to form tubes in the presence or absence of test extracts and compounds may be determined using a capillary tubule disruption assay with pre-formed primary human umbilical vein endothelial cells (HUVEC) in a cell culture system.

Briefly, HUVECs are cultured in vitro on Extracellular Matrix, which stimulates the attachment and tubular morphogenesis of endothelial cells to form capillary-like lumen structures. These in vitro formed capillary tubules are similar to human blood vessel capillaries in many aspects. The capillary tube assay is based on this phenomenon and is used for evaluation of potential vasculature targeting agents.

HUVEC cultures are grown in a 5% CO₂ 37° C. cell incubator. The full growth medium for HUVECs is Endothelial Cell Basal Medium (EBM) supplemented with 2% fetal bovine serum (FBS), 12 μg/ml bovine brain extract, 1 μg/ml hydrocortisone, and 1 μg/ml GA-1000 (gentamicin-amphothericin). HUVEC cultures between passage 3 and 8 may be used for all assay experiments.

HUVECs are pre-labeled with fluorescent agent Calcein AM and seeded in Extracellular Matrix coated 96-well culture plate with their full growth medium. After about four hours of the morphogenesis process, the endothelial capillary tubes should be formed. Then, test agent in designed doses in 50 μl volume is applied into the formed capillary tubule cultures as treatment conditions. The no-treatment controls can be added with vehicle of test agents. Sutent, a FDA approved anti-angiogenic drug one concentration can be included as assay performance control. After about six hours of treatment, the endothelial tubule morphology in each well is examined by microscopy, imaged, and the capillary disrupting activities under treatment conditions can be quantitatively analyzed. Each test conditions can be conducted in duplicate wells, including controls.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of sanitizing and hydrating skin, the method comprising topically applying to skin of a person a composition comprising an effective amount of alcohol, glycerin, betaine, PEG-50 shea butter, butylene glycol, and Aloe barbadensis leaf extract, wherein topical application of the composition sanitizes and hydrates skin.

2. The method of claim 1, wherein topical application of the composition to the skin of a person reduces one or more microorganism populations present on the skin by at least 99.9% within 15 seconds of application of the composition.

3. The method of claim 2, wherein the one or more microorganism populations comprise Haemophilus influenza, Streptococcus pneumoniae, Bacteroides fragilis, Pseudomonas aeruginosa, Campylobacter jejuni, Streptococcus pyogenes, Enterobacter cloacae, Burkholderia cepacia, Escherichia coli, Klebsiella pneumoniae, Serratia marcescens, Salmonella enterica, Shigella sonnei, Enterococcus faecalis, Staphylococcus aureus, methicillin-resistant Staphylococcus aureus, and/or Listeria monocytogenes.

4. The method of claim 1, wherein the composition comprises 60 to 80% by weight of alcohol, 0.1% to 15% by weight of glycerin, 0.1% to 10% by weight of betaine, 0.001% to 1% by weight of PEG-50 shea butter, 0.001 to 1% by weight of butylene glycol, and 0.001 to 1% by weight of Aloe barbadensis leaf extract.

5. The method of claim 1, wherein the composition comprises an effective amount of alcohol to sanitize skin and/or an effective amount of glycerin, betaine, PEG-50 shea butter, butylene glycol, and Aloe barbadensis leaf extract to hydrate skin.

6. The method of claim 1, wherein the alcohol comprises ethanol and/or denatured alcohol.

7. The method of claim 1, wherein the composition further comprises one or more of: a humectant, a skin conditioning agent, a viscosity controlling agent, a preservative, and/or a pH adjuster.

8. The method of claim 1, wherein the composition further comprises water and/or phenoxyethanol.

9. The method of claim 8, wherein the composition comprises 60 to 80% by weight of alcohol, 10% to 40% by weight of water, 3% to 7% by weight of glycerin, 0.5% to 1.5% by weight of betaine, 0.01% to 0.1% by weight of PEG-50 shea butter, 0.01 to 0.1% by weight of butylene glycol, 0.005 to 0.05% by weight of Aloe barbadensis leaf extract, and 0.0001% to 0.1% by weight of phenoxyethanol.

10. The method of claim 1, wherein the composition consists of 60 to 80% by weight of alcohol, 10% to 40% by weight of water, 3% to 7% by weight of glycerin, 0.5% to 1.5% by weight of betaine, 0.01% to 0.1% by weight of PEG-50 shea butter, 0.01 to 0.1% by weight of butylene glycol, 0.005 to 0.05% by weight of Aloe barbadensis leaf extract, and 0.0001% to 0.1% by weight of phenoxyethanol.

11. A topical sanitizing and hydrating composition comprising an effective amount of a combination of alcohol, glycerin, betaine, PEG-50 shea butter, butylene glycol, and Aloe barbadensis leaf extract to sanitize skin and/or hydrate skin.

12. The composition of claim 11, wherein the composition is capable of reducing one or more microorganism populations present on the skin by at least 99.9% within 15 seconds of application of the composition to the skin.

13. The composition of claim 12, wherein the one or more microorganism populations comprise Haemophilus influenza, Streptococcus pneumoniae, Bacteroides fragilis, Pseudomonas aeruginosa, Campylobacter jejuni, Streptococcus pyogenes, Enterobacter cloacae, Burkholderia cepacia, Escherichia coli, Klebsiella pneumoniae, Serratia marcescens, Salmonella enterica, Shigella sonnei, Enterococcus faecalis, Staphylococcus aureus, methicillin-resistant Staphylococcus aureus, and/or Listeria monocytogenes.

14. The composition of claim 11, comprising 60 to 80% by weight of alcohol, 0.1% to 15% by weight of glycerin, 0.1% to 10% by weight of betaine, 0.001% to 1% by weight of PEG-50 shea butter, 0.001 to 1% by weight of butylene glycol, and 0.001 to 1% by weight of Aloe barbadensis leaf extract.

15. The composition of claim 11, wherein the alcohol comprises ethanol and/or denatured alcohol.

16. The composition of claim 11, further comprising one or more of: a humectant, a skin conditioning agent, a viscosity controlling agent, a preservative, and/or a pH adjuster.

17. The composition of claim 11, further comprising water and/or phenoxyethanol.

18. The composition of claim 17, comprising 60 to 80% by weight of alcohol, 10% to 40% by weight of water, 3% to 7% by weight of glycerin, 0.5% to 1.5% by weight of betaine, 0.01% to 0.1% by weight of PEG-50 shea butter, 0.01 to 0.1% by weight of butylene glycol, 0.005 to 0.05% by weight of Aloe barbadensis leaf extract, and 0.0001% to 0.1% by weight of phenoxyethanol.

19. The composition of claim 11, wherein the composition consists of 60 to 80% by weight of alcohol, 10% to 40% by weight of water, 3% to 7% by weight of glycerin, 0.5% to 1.5% by weight of betaine, 0.01% to 0.1% by weight of PEG-50 shea butter, 0.01 to 0.1% by weight of butylene glycol, 0.005 to 0.05% by weight of Aloe barbadensis leaf extract, and 0.0001% to 0.1% by weight of phenoxyethanol.

20. A method of sanitizing skin and/or hydrating skin, the method comprising topically applying to skin of a person a composition of claim 11, wherein topical application of the composition sanitizes skin and/or hydrates skin.
